(19) United States
(12) Patent Application Publication
(10) Pub. No.: US 2002/0137917 A1
(43) Pub. Date: Sep. 26, 2002

(54) Assay for perkinsus in shellfish

(75) Inventors: Gerardo R. Vasta, Baltimore, MD (US); Adam G. Marsh, Milton, DE (US); Jos?eacute; A. Fernández-Robledo, Baltimor, MD (US); Cathleen A. Coss, Hagerstown, MD (US); Anita C. Wright, Gainesville, FL (US)

Correspondence Address: Steven J. Hultquist, Intellectual Property/Technology Law, P.O. Box 14329, Research Triangle Park, NC, 27709, US

(73) Assignee: University of Maryland Biotechnology Institute, Baltimore, MD

(21) Appl. No.: 09/771,935

(22) Filed: Jan. 30, 2001

Related U.S. Application Data
(63) Continuation-in-part of application No. 08/900,117, filed on Jul. 25, 1997.
(60) Provisional application No. 60/023,345, filed on Jul. 26, 1996.

(51) Int. Cl. C07H 21/04; C12Q 1/68

(52) U.S. Cl. 536/23.7; 536/24.3; 435/6

(57) ABSTRACT

The present invention is directed to oligonucleotides used as amplification primers and assay probes for species-specific detection and identification of the protozoan Perkinsus in shellfish. The oligonucleotides are designed to preferentially hybridize to what has been found to be a species-unique sequence in the target organism's genome. Preferential hybridization means, for example, that the inventive primers amplify the target sequence in P. marinus with little or no detectable amplification of target sequences of other species of protozoa such as P. atlanticus thereby making the assay species specific.

### CROSS-REFERENCE TO RELATED APPLICATION

This is a continuation-part of U.S. patent application Ser. No. 08/900,117 filed on Jul. 25, 1997 and which is a continuation-in-part of U.S. Provisional application No. 60/023,345 filed on Jul. 26, 1996, the contents of which are incorporated herein.

### FEDERAL SPONSORSHIP OF INVENTION

The U.S. Government has a paid-up license in this invention as provided for by the terms of agreement number NA47FL-0163 NOAA/NMFS awarded under the Oyster Disease Research Program by the National Oceanographic and Atmospheric Administration of the U.S. Department of Commerce.

### FIELD OF THE INVENTION

The present invention relates to diagnostic assays and more particularly to diagnostic assays which utilize the polymerase chain reaction (PCR) to detect the presence and concentration of a pathogen suspected of infecting shellfish.

### BACKGROUND OF THE INVENTION

A. Diseases in Shellfish (oysters, clams, and other bivalves)

Shellfish, particularly oysters, are universally recognized as important sources of commercially valuable food and as organisms that play important roles in the aquatic ecosystem as part of the food chain and in reducing the turbidity of water through filtration. Unfortunately, protozoan, bacterial, fungal and viral epizootic diseases are destroying massive numbers of natural and cultivated stocks of oysters and other shellfish in coastal areas of the United States. A clear example of the serious impact of shellfish diseases is the enormous decline in oyster production from the Chesapeake Bay. Oyster production has plummeted from a high of 2.5 million bushels harvested annually in the early 1980's to less than 1% of this level in the past few years.

Protozoan infections is a primary cause of mass mortality of the eastern oyster Crassostrea virginica along the Gulf of Mexico and Atlantic coasts. The major disease is “Dermo,” caused by the endoparasitic protozoan Perkinsus marinus. This disease, for which there is no known remedy, has resulted in a critical reduction of existing populations and is a major cause of the collapse of the oyster industry in the Chesapeake Bay. The range of this parasite has now extended into low salinity areas of Chesapeake Bay tributaries that are sources of oyster seed stock. Additionally, the parasite has been detected in North Atlantic waters from Delaware Bay to Maine that were previously disease free and thought to be uninfected due to cold water conditions.

Other Perkinsus species have been detected in mollusks around the world and cause mass mortalities in commercially important shellfish from Australia and Europe. In addition to P. marinus, other pathogenic species include P. olseni in the abalone in Australia and P. atlanticus in the clam in Europe.

The transplantation of brood and seed stocks between countries has become a frequently used alternative to raising native shellfish. However, this practice can also lead to the spread of disease and the destruction of native stocks because of the lack of appropriate diagnostic tests. Frequently, natural resource managers seek to introduce non-indigenous oysters having desirable characteristics to their aquatic jurisdiction. However, if the introduced species carries Dermo or other infectious diseases the consequences can be devastating.

B. Currently Available Shellfish Dermo Disease Detection Methods

The continuing decline of oyster stocks as a result of Dermo and other diseases has created a demand for new technologies to efficiently detect and monitor these diseases in indigenous and transplanted oysters. The most significant obstacle to developing effective treatment and management strategies for controlling P. marinus infections is the lack of a sensitive assay that would allow for both the detection of P. infections is the lack of a sensitive assay that would allow for both the detection of P. marinus at low infection levels and discrimination between putative geographic subpopulations of P. marinus as well as other Perkinsus species. There is a need for sensitive and specific diagnostic assays for P. marinus to detect, for example, cryptic infections in oyster seed-stocks, latent infections in overwintering oyster populations, the onset of infection in oyster larvae and spat, the presence of P. marinus in other marine organisms that may serve as secondary vectors or reservoirs, and the genetic structure of parasite field populations.

The life cycle of P. marinus within the host consists of an intracellular vegetative state (trophozoite) which proliferates by multiple fusion and/or budding. Mature trophozoites enlarge to become prezoosporangia, which upon entering the water column sporulate to release large numbers of biflagellated zoospores. These motile zoospores presumably give rise to trophozoites once they infect oyster tissue, but the mechanism of infection is unknown. With most prior art detection methods only trophozoites can be detected in most host tissues but not the other stages. It would be desirable to have an assay that is sensitive enough to detect any P. marinus life stage present in a sample.

Histology was the first technique used for diagnosis of Perkinsus marinus. The fluid thioglycollate media (FTM) assay (Ray,1952,1966) which has been the routine method for Perkinsus species diagnosis was adopted because it was inexpensive and simple to perform. In the FTM assay oyster tissue is incubated with antibiotic-fortified medium under conditions in which parasites at the trophozoite stage enlarge into hypnospores. These stain with Lugol's iodine solution for visualization of the parasite as a blue-dark sphere (Ray, 1966). The FTM assay relies on the enlargement of the trophozoites into hypnospores in fluid thioglycollate medium, a feature shared by all Perkinsus species and so does not distinguish between them. Hence this assay is not species specific. Consequently, most studies on Perkinsus from bivalves refer to them as Perkinsus species because no specific identification is possible. In addition, this assay is only able to detect one stage in the lifecycle of these parasites and takes between 4 and 7 days to complete. Hence, effective diagnosis in terms of sensitivity, species-specificity, and rapidity are needed for appropriate management of bivalve resources.

Antibody-based assays for the detection of P. marinus proteins in oyster tissues have recently been used with mixed success due to lack of sensitivity (Choi et al., 1991; Dungan and Roberson, 1993). These antibodies were raised against only one life stage of this parasite. Consequently the lack of sensitivity may be due to changes in epitope expression by the parasite at different life cycle stages. Also, a general feature of parasites is their ability to modify their epitope expression over time making an antibody-based assay unreliable. Because of these disadvantages, this technique never became established as a routine diagnostic assay for Perkinsus.

The sensitivity of PCR for detection of trace quantities of foreign DNAs in heterogenous samples has made this technology an ideal choice for identifying infectious agents and has been used with great success to screen protozoan pathogens in aquaculture (Cai et al., 1992; Stokes and Burreson, 1995). Different gene regions have been used as PCR targets. The ability of a PCR assay targeting DNA to distinguish between genetically related species and subspecies depends on the correct choice of a gene target. Fong et al. (1993) suggested the use of the small subunit of the rRNA gene of Perkinsus to design probes for this parasite, however this region cannot be used as a PCR target because the high degree of sequence identity that exists in homologous genes among between this parasite and its host.

The introduction and transplantation of shellfish has contributed to the spread of disease. The Working Group on Diseases of the International Council for the Exploration of the Seas (ICES) has established criteria for the introduction of exotic species as well as for transferred species. These criteria require periodic inspection and testing of the material using state of the art techniques before the mass transplantation and during quarantine. In addition, a significant obstacle to developing effective treatment and management strategies for controlling P. marinus infections in oysters is identifying when exactly an infection begins and the source of the pathogen. The only diagnostic technique routinely used up to this point has been the FTM assay which, as described, lacks the necessary requirements of sensitivity and specificity in detection of the parasite in order to help guarantee disease-free oysters.

There is a strong need, therefore, for a diagnostic assay that is (1) sensitive enough to detect the presence of the various species of Perkinsus at low levels, and (2) specific enough to discriminate between putative geographic races or strains of P. marinus and between the various species of Perkinsus, and (3) that can be completed rapidly enough to provide resource managers with timely information about the disease status of oyster populations, especially of oysters proposed for introduction from distant sources.

### SUMMARY OF THE INVENTION

The present invention is directed to oligonucleotides used as amplification primers and assay probes for species-specific detection and identification of the protozoan Perkinsus in shellfish. The oligonucleotides are designed to preferentially hybridize to what has been found to be a species-unique sequence in the target organism's genome. Preferential hybridization means, for example, that the inventive primers amplify the target sequence in P. marinus with little or no detectable amplification of target sequences of other species of protozoa such as P. atlanticus thereby making the assay species specific.

The polymerase chain reaction (“PCR”) and other probe based assays require a specific DNA sequence as a target. In diagnostic applications it is desirable that the DNA target sequence have a high copy number so as to increase the likelihood of detection at low levels of infection. Most organisms contain multiple copies of the regions that code for the ribosomal RNAs (“rRNA”). Usually RNA genes are organized in clusters comprising the following sequences shown schematically in FIG. 1: 5.0S region, non-transcribed spacer (“NTS”), small subunit (“SSU”) region, internal transcribed spacer 1 (“ITS1”), 5.8S gene, internal transcribed spacer 2 (“ITS2”), and large subunit (“LSU”) region. The NTS separates transcription units but is not represented in the mature RNA products. Although this part of the molecule may not be important in terms of virulence or parasite proliferation, it is used, in accordance with the present invention, as a marker to distinguish between species and types. Coding regions of the rRNA genes are evolutionarily conserved, whereas the NTS is more variable and can differ significantly between even closely related species. The amplification primers and probes of the invention are based on the NTS domain of P. marinus and other species of this genus. Since each eukaryotic microorganism has its own unique, species specific NTS sequence, the assay according to the present invention can be used to detect the genomic “fingerprint” of any target microorganism in a sample being tested. In short, to create an assay for a particular microorganism one needs to (i) isolate and sequence the NTS region for that species, and (ii) design an oligonucleotide probe or primers that will preferentially hybridize to the unique NTS.

These techniques were employed in the examples provided herein to identify the sequences of P. marinus, P. atlanticus, and P. andrewsi. Primers were then designed for each of these NTS and a PCR based assay was conducted on tissue removed from shellfish. The assay succeeded in detecting P. marinus, P. atlanticus and P. andrewsi in shellfish tissues and body fluids thereby providing valuable information about infection status.

By using the primers disclosed herein in PCR amplification, genetic variability within P. marinus can also be detected. Distinction between two different types of P. marinus DNA has been discovered. We refer to these types as Type I and Type II DNA and therefore these primers constitute the preferred method for determination of the presence of P. marinus.

The inventive assay has distinct advantages over the routine methods used presently. This assay can be performed in several hours rather than the 4 to 7 days required of prior art assay. The inventive assay is expected to become even more rapid as DNA technology improves. Another advantage is that the assay is sensitive enough to detect even a single parasite cell.

The assay according to the present invention thus provides (a) a rapid and economical assay that can be implemented in most labs with little in the way of specialized equipment; (b) a species-specific assay that can provide genetic lineage information about a particular Perkinsus sample; and (c) a sensitive assay for the detection of Perkinsus in tissues, body fluids, spat, and environmental samples. The inventive assay is also useful, for example, in helping marine biologists learn how P. marinus infects C. virginica because treatment approaches are dependent on identifying the life stage of the oyster that is the most susceptible to parasite entry and whether C. virginica populations are challenged by one continuous population of P. marinus or by discreet geographical races in order for management strategies to be implemented for regional areas infected by discrete P. marinus populations.

In a preferred embodiment the assay incorporates the PCR for the detection of DNA from P. marinus in oyster tissues. It has been found to be both sensitive enough to detect the presence of P. marinus at low levels in juvenile oysters and spat and specific enough to discriminate between different geographic races or strains of P. marinus. The invention may be employed both in commercial aquaculture as well as in areas of marine biology research where sensitive and reliable detection method are crucial in studying the etiology of diseases in populations of oysters and other shellfish.

The assay according to the present invention has been employed to distinguish two sequence types of P. marinus. This variability may reflect different P. marinus types or races as well as a new way to define the parasite distribution. Two new sets of primers were developed based on the difference between the P. marinus types found. The primers serve as tools for a PCR reaction specific for the two types of P. marinus.

It is believed that the persistence of P. marinus in areas of the East coast where the salinity is low may reflect the existence of P. marinus races tolerant to low salinity. Hence the assay according to the present invention also provides valuable information about aspects of P. marinus types that helps marine biologists understand why one type prevail over the other in one particular area. Parasite genotypes and phenotypes may also reflect a different host susceptibility to the parasite. Hence, characterization of Perkinsus types would permit to improve the management strategies because a more effective control of the pathogen could be established for each particular region depending the parasite type more prevalent.

In addition to P. marinus, Perkinsus-like organisms of unknown virulence have been detected in bivalve species sympatric with C. virginica (Andrews 1954). For example, production of zoospores seawater without preincubation in FTM (Kleinschuster and Swink 1993; Perkins 1988), morphological features of the trophozoite in host tissue (Perkins 1988), and partial sequences of the SSU and ITS regions reported earlier (Coss et al. 1997) indicated that Perkinsus isolates from the baltic clam Macoma balthica may be a different species from P. marinus. The identity and host specificity of the various Perkinsus species described in sympatric mollusk species in Chesapeake Bay, and the possibility that these may constitute alternate hosts or reservoir species for P. marinus has received limited attention (Coss et al. 1997; McLaughlin and Faisal 1998; Coss, Robledo, and Vasta, in press). Accordingly, questions about the presence of P. marinus in non-oyster bivalves, as well as other Perkinsus species infecting C. virginica, are addressed in this application.

The study reported herein was designed to: (1) characterize the rRNA locus of Perkinsus sp. from M. balthica, hereafter referred to as Perkinsus andrewsi n. sp, for comparison with rRNA sequences reported for known Perkinsus species; (2) determine the range of intraspecific variability in regions of the rRNA of P. andrewsi n.sp.; (3) develop and validate a PCR-based assay based on the NTS (non-transcribed spacer) sequence for diagnostic of P. andrewsi n.sp.; (4) examine field samples of clams (M. balthica, M. mitchelli, and M. mercenaria) and oysters (C. virginica) for the presence of P. andrewsi n.sp. Using the specific PCR assay; and (5) assess the presence of P. marinus, by use of a specific PCR-based assay (Marsh, Gauthier, and Vasta 1995; Robledo et al. 1998) in sympatric clams and oysters. This study complements the ultrastructural characterization of this in vitro clonally-propagated Perkinsus species from M. balthica and its comparison to the previously described Perkinsus species (Coss, Robledo, and Vasta, in press).

The rRNA of Perkinsus atlanticus from the clam Ruditapes decussatus cultivated on the Atlantic coast of Spain was cloned and sequenced. Sequences of the internal transcrbed spacer (ITS) form the rRNA locus were compared to sequences reported earlier for a P. atlanticus isolate from Portugal and those from the Perkinsus species. The ITS1 sequence of the Spanish P. atlanticus isolate was identical to the Portuguese P.atlanticus sequence and had 76.6% identity to the P. marinus ITS2, and 99.5% identity to the P. olseni ITS2.

Based on the NTS sequence of P. atlanticus from Spain and the differences with P. marinus NTS (62.2% identity) the PCR-based diagnostic assay was utlized with a lowest limit of detection of 0.01 amol of cloned NTS DNA as assessed on ethidium bromide-stained agocrose gels. Specitity of the PCR-based assay was tested with samples from the clams R. decussatus, Ruditapes philippinarium, and Venerupis pullastra collected in P. atlanticus-enzootic areas of Spain. The specitity and sensitivity demonstrated for this NTS-based PCR assay validate its use as a tool for assessment of P. atlanticus in molluscs.

Additonally, a set of “generic” primers (forward primer PER1-5′TAG TAC CCG CTC ATT GTG G-3′- and the reverse primer PER 2-5′TGC AAT GCT TGC GAG CT 3′) based on NTS sequences aimed to amplify DNA from both P. marinus and Perkinsus sp. from M. balthica, (P. andrewsi) was designed and the PCR assay's performance examined. The “generic” primers also amplify P. atlanticus DNA.

The amplified DNA target provides sequence information of the genome and can therefore be secondarily employed to distinguish between related genetic strains of a pathogen if the DNA target region is carefully selected.

With the foregoing and other objects, advantages and features of the invention that will become hereinafter apparent, the nature of the invention may be more clearly understood by reference to the following detailed description of the invention, the appended claims and to the several views illustrated in the figures.

### BRIEF DESCRIPTION OF THE DRAWINGS

FIG. 1 is a schematic diagram of an rRNA gene cluster.

FIG. 2 is the nucleotide sequence of the nontranscribed space (NTS) from Perkinsus marinus.

FIG. 3 is the nucleotide sequence of the NTS of rRNA of P. andrewsi, isolated from Macoma balthica.

FIG. 4 is the nucleotide sequence of the NTS of rRNA of Perkinsus mackini isolated from Mercenaria mercenaria.

FIG. 5 lists representative sets of primers used for diagnosis of Perkinsus marinus, Perkinsus andrewsi, and primers for Perkinsus marinus typing.

FIG. 6 is an agarose gel electrophoresis of amplified products of PCR demonstrating species-specificity of P. marinus diagnostic primers. Amplification of DNA with P. marinus diagnostic primers (d) only occurred with P. marinus samples. However, PCR with actin primers (a) amplified all samples. P. sp. (1) Perkinsus sp. from Anadara trapezia, P. o. P. olseni from Haliotis laeviagata, P.a. P. atlanticus from Ruditapes decussatus, P.m. P. marinus from Crassostrea virginica. M. 123 bp DNA ladder.

FIG. 7 are agarose gel electrophoresis of amplified products of PCR demonstrating the sensitivity of P. marinus diagnostic primers. Using this methodology as few as one cell was detected. Three samples (×3) were used for 1,2,5,8, and 10 cells.

FIG. 8 is an agarose gel electrophoresis of PCR products of different Perkinsus isolates using P. marinus diagnostic primers (a) and primers derived Perkinsus sp. isolated Macoma balthica (b). 1. Perkinsus sp. from Mercenaria mercenaria 2. Perkinsus sp. from Macoma balthica, 3. P. marinus from Crassostrea virginica, 4. Negative controls, M. 123 bp DNA ladder.

FIG. 9 is amplification of the Perkinsus marinus DNA target using a known amount of total P. marinus DNA in a 10× serial dilution with a constant level of oyster genomic DNA (1 μg/μl). A. Ethidium bromide visualization of the resolving gel. B. Southern blot of the above gel. C. Dot-blot hybridization of PCR amplification.

FIG. 10 is a ribosomal DNA nucleotide sequences of the non-transcribed spacer (NTS) domain from Perkinsus marinus Type I and Type II. Nucleotides shown in boldface indicate differences between P. marinus types.

FIG. 11 is an ethidium bromide stained agarose gel electrophoresis of PCR products generated by amplification of DNA derived from oysters (Crassostrea virginica) infected with Perkinsus marinus. Lanes 1 to 5 using primers PM5/PM7 specific for P. marinus type 1 (lane a) and primers PM6/PM8 specific for P. marinus type II (lane b). M. 123 bp DNA ladder. (+) control P. marinus type II. Note the presence of bands corresponding to both Perkinsus types in the same oyster in samples #2 and #3.

FIG. 12 is an agarose gel showing the patterns of Perkinsus marinus types after Spe 1 digestion of PCR amplified products. P. marinus type I (samples #1 and #2) and P. marinus type II (sample #3). Sample with enzyme (lane a). Sample without enzyme (lane b).

FIG. 13 is a chart showing the distribution of Perkinsus marinus types in samples form Maryland, Florida, and Louisiana.

FIG. 14 is a chart showing the standard curves of the dot blot and Southern blot of the amplified Perkinsus marinus DNA target as a function of total P. marinus DNA that was used in the amplification.

FIG. 15 is an agarose gel electrophoresis of amplified products of PCR demonstrating the presence of Perkinsus marinus is samples obtained from the mantle of Crassostrea virginica from Louisiana (+): positive control, (−) negative control.

FIG. 16 is an agarose gel electrophoresis of amplified products of PCR demonstrating the presence of Perkinsus marinus in samples obtained from Macoma balthica from Rhode River. Lanes 1-7: DNA from M. balthica individuals, (+): positive control, (−): negative control.

FIG. 17 is the nucleotide sequences of the NTS of rRNA of P. atlanticus.

FIGS. 18a-b is the nucleotide sequence of the SSU rRNA of P. andrewsi.

FIG. 19 is the nucleotide sequence of the ITS1-5.8.S-ITS2 regions of P. andrewsi.

FIGS. 20-21 lists representative sets of primers for P. marinus, P. andrewsi, P. makinus typing and “generic primers”.

### REFERENCES

Ausubel, F. M., R. Brent, R. E. Kingston, D. D. Moore, J. G. Seidman, J. A. Smith and K. Struhl (eds.). 1992. Short Protocols in Molecular Biology, 2nd Edition. John Wiley and Sons, NY.

Cai, J., Collins, M. D., McDonald, V., and Thompson, D. E. 1992. PCR Cloning and Nucleotide Sequence Determination of the 18S rRNA Genes and Internal Transcribed Spacer I of the Protozoan Parasites Cryptosporidum parvum and Cryptosporidium muris. Bioch. Bioph. Act. 1131:317-320.

Choi, K. S., D. H. Lewis, E. N. Powell, P. F. Frelier, and S. M. Ray. 1991. A polyclonal antibody developed from Perkinsus marinus hypnospores fails to cross react with other life stages of P. marinus in oyster (Crassostrea virginica) tissues. J. Shellfish Res. 10: 411-415.

Dieffenbach, C. W. and Dveksler, G. S. 1995. PCR Primer: A Laboratory Manual. Cold Spring Harbor Laboratory Press, New York

Dungan, C. F. and B. S. Roberson. 1993. Binding specificities of mono- and polyclonal antibodies to the protozoan oyster pathogen Perkinsus marinus. Dis. aquat. Org. 15: 9-22.

Fong, D., R. Rodriguez, K. Koo, J. Sun, M. Sogin, D. Bushek, D. T. L. Littlewood, and S. Ford. 1993. Small subunit ribosomal RNA gene sequence of the oyster parasite Perkinsus marinus. Mol. Mar. Biol. and Biotech. 2: 3436-350.

Goggin, C. L. 1994. Variations in the Internal Transcribed Spacers and 5.8S Ribosomal RNA from Five Isolates of the Marine Parasite Perkinsus (Protista, Apicomplexa). Mol. Biochem. Parasitol. 65: 179-182.

Innis, M., D. Gelfand, J. Sninsky and T. White (eds.). 1990. PCR protocols: A guide to methods and applications. Academic Press Inc., NY, N.Y.

Mackin, J. G. 1962. Oyster disease caused by Dermocystidium marinum and other microorganisms in Louisiana. Publ. Inst. Mar. Sci. Univ. Tex 7:132-229.

Marsh, A. G., J. D. Gauthier, G. R. Vasta. 1995. A semiquantitative PCR assay for assessing Perkinsus marinus infections in the eastern oyster, Crassostrea virginica. J. Parasitol., 81: 577-583.

Perkins, F. O. 1996. Forward. J. Shellfish Res. 15:5-7

Ray, S. M. 1966. A review of the culture method for detecting Dermocystidium marinum, with suggested modifications and precautions. Proc. Natl. Shellfish. Assoc. 54: 55-69.

Sambrook, G., Fartsch, E. F., Maniatis, T. 1989. Molecular Cloning, A Laboratory Manual, Second Edition. Cold Spring Harbor Laboratory, cold Spring Harbor, N.Y.

Sindermann, C. J. and Lihgtner, D. V. 1988. Disease Diagnosis and Control in North American Marine Aquaculture. Elsevier, N.Y., 431 pp.

Stokes, N. A. and Burreson, E. M. 1995. A Sensitive and Specific DNA Probe for the Oyster Pathogen Haplosporidium Nelsoni. J. Euk. Microbiol. 42: 350-357.

Sykes, P. J., S. H. Neoh, M. J. Brisco, E. Hughes, J. Condon, and A. A. Morley. 1992. Quantitation of targets for PCR by use of limiting dilution. Biotechniques 13: 444-449.

### DETAILED DESCRIPTION OF THE INVENTION

A. Definitions

The following terms are defined herein as follows:

“DNA amplification” as used herein refers to any process which increases the number of copies of a specific DNA sequence. A variety of processes are known. One of the most commonly used is the Polymerase Chain Reaction (PCR) process of Mullis as described in U.S. Pat. Nos. 4,683,195 and 4,683,202 both issued on Jul. 28, 1987. In general, the PCR amplification process involves an enzymatic chain reaction for preparing exponential quantities of a specific nucleic acid sequence. It requires a small amount of a sequence to initiate the chain reaction and oligonucleotide primers which will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed. In the present invention the amplification results in an extension product of one sequence localized between two genes. Since these genes are multiple copy and the sequence target is between each copy, there will be exponential amplification for each of the copies. The extension products sizes using discrete primers will provide a specific fingerprint for each microorganism.

“Primer” means an oligonucleotide comprised of more than three deoxyribonucleotide used in amplification. Its exact length will depend on many factors relating to the ultimate function and use of the oligonucleotide primer, including temperature, source of the primer and use of the method. The primer can occur naturally (as a purified fragment or restriction digestion) or be produced synthetically. The primer is capable of acting as an initiation point for synthesis, when placed under conditions which induce synthesis of a primer extension product complementary to a nucleic acid strand. The conditions can include the presence of nucleotides and an inducing agent such as a DNA polymerase at a suitable temperature and annealing and extension times as well as the appropriate buffer (pH, magnesium chloride (MgCl₂) and potassium chloride (KCl₂) concentrations, and adjuncts). In the preferred embodiment the primer is a single-stranded oligodeoxyribonucleotide of sufficient length to prime the synthesis of an extension product from a specific sequence in the presence of an inducing agent. In the present application in the preferred embodiment the oligonucleotides are usually between about 10 mer and 35 mer. In the most preferred embodiment they are between 17 and 24 mer. Sensitivity and specificity of the oligonucleotide primers are determined by the primer length and uniqueness of sequence within a given sample of a template DNA. Primers which are too short, for example, less than 10 mer may show non-specific binding to a wide variety of sequences in the genomic DNA and thus are not very helpful. Each primer pair herein is selected to be substantially complementary to the different strands of each specific NTS region to which the primer pairs bind. Thus one primer of each pair is sufficiently complementary to hybridize with a part of the sequence in the sense strand and the other primer of each pair is sufficiently complementary to hybridize with a different part of the same repetitive sequence in the anti-sense strand.

“Oligonucleotide” as used herein means any nucleotide of more than 3 bases in length used to facilitate detection or identification of a target nucleic acid, including primers.

“Species-specific” means detection, amplification or oligonucleotide hybridization in a species of organism or a group of related species without substantial detection, amplification, or oligonucleotide hybridization in other species of the same genus or species of a different genus.

“Stringent annealing conditions” means that in those conditions the specificity, efficiency and fidelity of the PCR amplification will generate one and only one amplification product that is the intended target sequence.

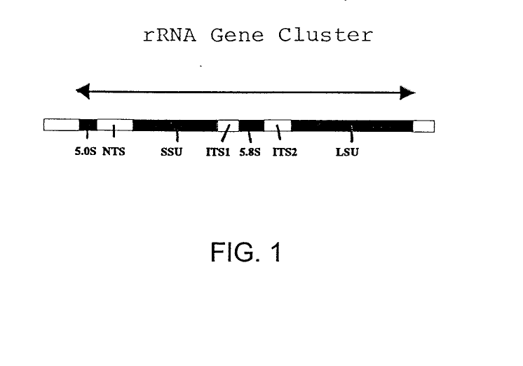

“Non-transcribed spacer” or “NTS” are sequences that separate different portions of the rRNA at the level of the gene. The NTS are typically located between the regions that code for the 5.0S and the Small Subunit (SSU). (FIG. 1)

“Hybridize” or “Preferentially Hybridize” means the joining of two single stranded nucleotide sequences that are about 80% or more complementary.

B. Methods of Making Oligonucleotides

To create an assay for a particular species of microorganism one needs to (i) isolate and sequence the NTS region for that species, and (ii) design an oligonucleotide probe or primers that will preferentially hybridize to the unique NTS.

More particularly, to isolate and sequence an NTS region for a target species the following steps would be employed. First, it is determined whether the 5.0S and small subunit rRNA sequences for the species have been published in either the scientific literature or databases such as GENBANK. If so, using this sequence information, PCR primers are designed which hybridize to the rRNA genes flanking the NTS sequence using techniques known in the art (such as those described PCR Primer: a Laboratory Manual (Dieffenbach et al. 1995). The NTS is then amplified. Depending on the particular primer sequences selected, the best PCR conditions (annealing temperatures, pH, adjuncts, extension times, cycle numbers, salt concentrations) can be determined using any of a variety of commercially available computer programs such as GENE JOCKEY™ II (Biosoft, Ferguson, Mo.). Amplified products are resolved by agarose gel electrophoresis in the presence of ethidium bromide, recovered from the gel, and cloned into a commercially available cloning vector system (pGEM-T Vector, Promega, Madison, Wis.). Recombinant plasmids are transformed into competent cells and selected following the manufacture's protocol. Isolation of plasmid DNA is carried out using the method of Sambrook et al. (1989). For each PCR product, several clones with inserts are sequenced to confirm the sequence using an available DNA sequencing method (Applied Biosystems 373 DNA Sequencer, Perkin Elmer, Foster City, Calif.).

Alternatively, universal primers can be used to amplify the rRNA genes and the PCR products used to screen a genomic library (Sambrook et al., 1989) to pull out clones containing part of the entire NTS sequence.

After the NTS is sequenced, PCR primers specific for the NTS may be designed with assistance of commercially available computer programs such as GENE JOCKEY™ II (Biosoft, Ferguson, Mo.). The criteria for selecting the region to be amplified within the NTS are the following: length, sequence composition and melting temperature, and the ultimate applications, as will be readily known to those skilled in the art. The length of the region of the NTS that is selected to be amplified depends on the PCR primers selected. This length can be from 50 bp to full length, but is preferably from about 100 bp to 1200 bp, and optimally between about 250 and 600 bp. PCR conditions (annealing temperatures, pH, adjuncts, extension times, cycle numbers, salt concentrations) are determined following prescribed protocols in standard manuals such as PCR Primer: a Laboratory Manual (Dieffenbach et al. 1995). Primer lengths can be between 10 and 35 bases long, are preferably between 15 to 25 bases long, but most preferably will be between 17 and 24 bases long. Primers are tested against the target organism, related species, and the host in the case of a parasite. Sensitivity can be determined using different dilutions of target DNA for the PCR assay. General information about PCR and the design of primers not described herein may be found in Sambrook et al. (1989).

According to the method of the present invention, the liquid mixture is used in the amplification cycle of the PCR method. The amplification cycle comprises steps of: (i) denaturing a double-strand DNA (for about 10 seconds to 2 minutes at about 90° C., to 95° C.) (ii) annealing the single-strand DNA with the first and second primers (for about 30 seconds to about 3 minutes at about 37° C. to 70° C., and (iii) extending a DNA by the DNA polymerase (for about 30 seconds to about 5 minutes at about 65° C. to 80° C.). In the present invention the above mentioned amplification cycle is repeated 10 to 60 times, preferably 20 to 40 times. In the final cycle it is preferable to extend the heating time of the step (iii) to about 5 to 10 minutes so as to complete the DNA synthesis.

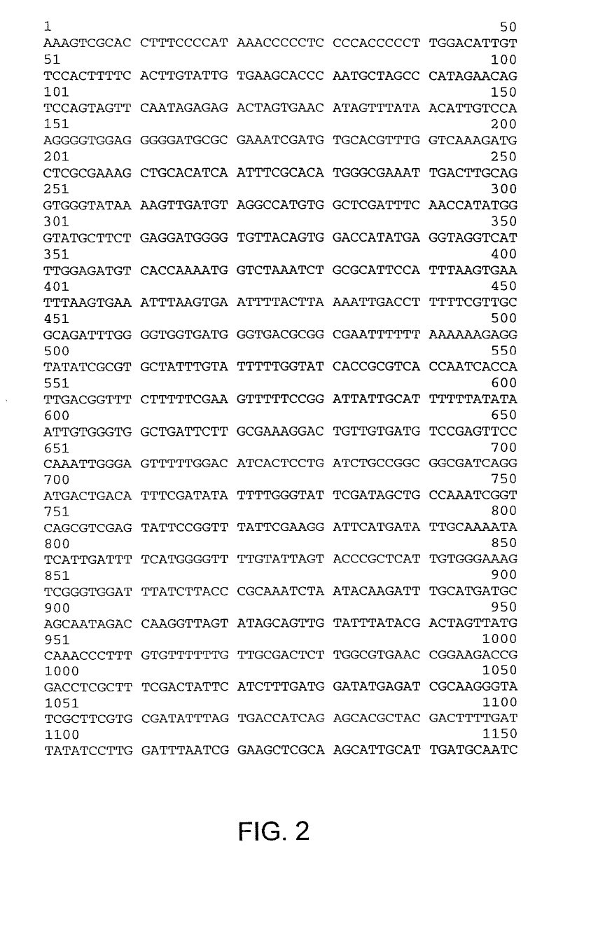

As described in the following Examples, the sequence of NTS region from an axenic culture of Perkinsus marinus has been cloned and sequenced and is shown in FIG. 2. Oligonucleotide primers to this DNA clone and evaluated their performance in detecting P. marinus infections in oyster tissues. Some details of our method may be found in the Publication, “A Semiquantitative PCR Assay for Assessing Perkinsus Marinus Infections in the Eastern Oyster, Crassostrea Virginica,” 1995, Journal of Parasitology 81(4):577-583, which is incorporated herein by reference. In this study, a 3.2 Kbp genomic clone of P. marinus was isolated and sequenced. A non-coding domain was identified and targeted for the development of a semiquantitative, polymerase chain reaction (PCR) assay for the presence of P. marinus in eastern oyster tissues. The assay involves extracting total DNA from oyster hemolymph and using 1 μg of that DNA as template in a stringent PCR amplification with oligonucleotide primers that are specific for the P. marinus NTS fragment. With this assay, it can detect 10 pg of total P. marinus DNA per 1 μg of oyster hemocyte DNA with ethidium bromide EtBr staining of agarose gels, 100 fg total P. marinus DNA with Southern Blot autoradiography, and 10 fg of total P. marinus DNA with dot blot hybridizations. This sensitive PCR assay has resulted in a method for estimating the level of P. marinus DNA in oyster hemolymph and it has been successfully applied to oyster gill tissues. The semiquantitative assay uses a dilution series to essentially titrate the point at which a P. marinus DNA target is no longer amplified in a sample. We refer to this technique as ‘Dilution EndPoint’ PCR. Using hemocytes obtained by withdrawing a 1 ml sample of hemolymph, this assay provides a non-destructive methodology for rapidly screening large numbers of adult oysters for the presence and quantification of P. marinus infection levels. Furthermore, we have now validated the PCR assay with field samples. When comparing PCR assay with the FTM assay the PCR technique is more sensitive and faster. Comparison of FTM and the PCR assays for P. marinus diagnosis showed that in 83% of the samples there was agreement between FTM and PCR analysis. Detailed analysis of the discrepancies showed that 15% of all samples were negative by FTM but positive by PCR analysis, while only 2% of the samples were FTM positive but were not amplified by PCR. The FTM−/PCR+ discrepancy may be attributed to a greater sensitivity of the PCR methodology. Using the same methodology P. marinus has been detected in four species of non-oyster bivalves in Chesapeake Bay. Consequently, this technique is applicable to other oyster and bivalve tissues (gills, mantle, rectum) and could potentially be applied to DNA extracts of whole larval or spat as well as sediment and water samples.

In a preferred embodiment sets of primers are used in PCR amplification. These sequences are derived from the nontranscribed sequence between the 5S and SSU rRNA genes of P. marinus. The entire nontranscribed sequence is shown in FIG. 2. Additional primers, of lengths greater or less than those described here, derived from this sequence could also function in the diagnostic test for P. marinus described herein.

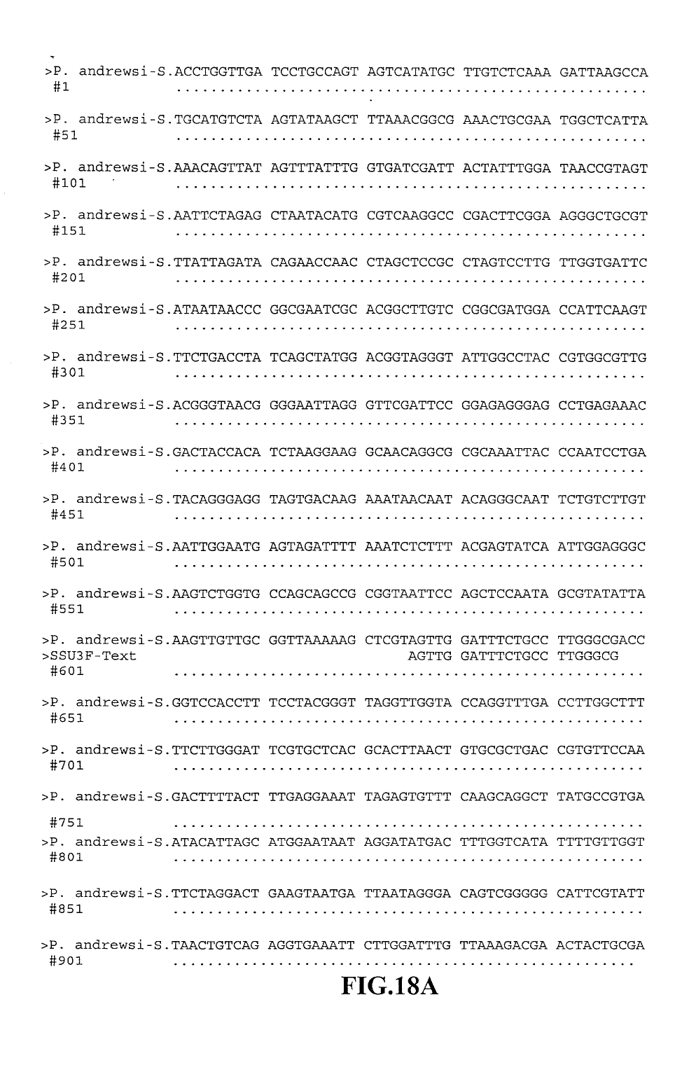

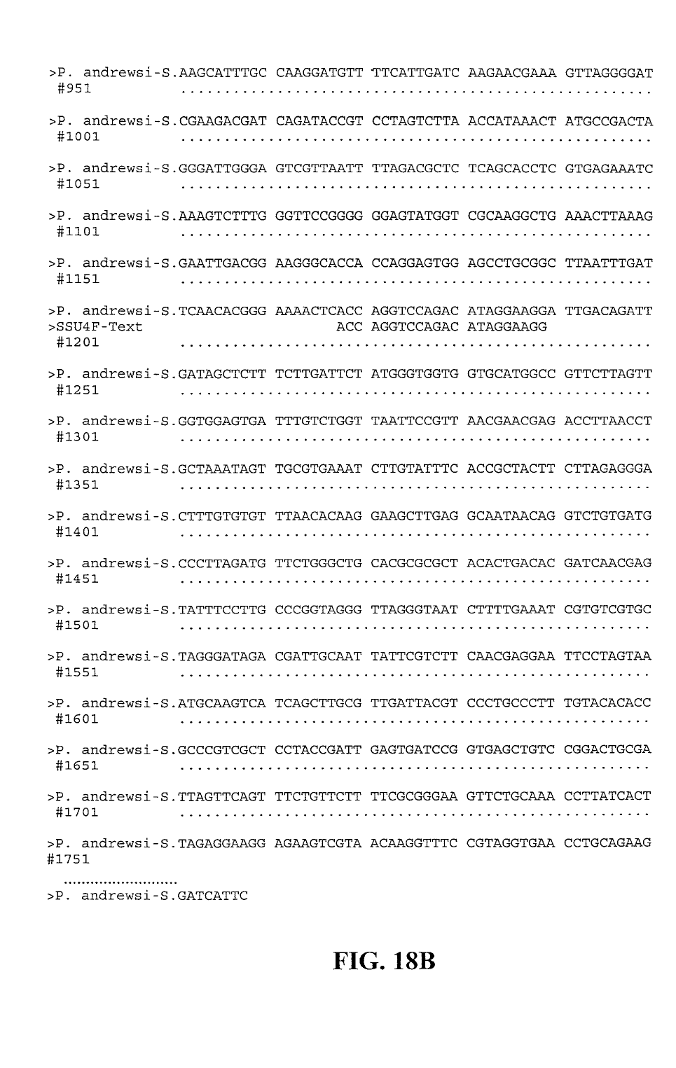

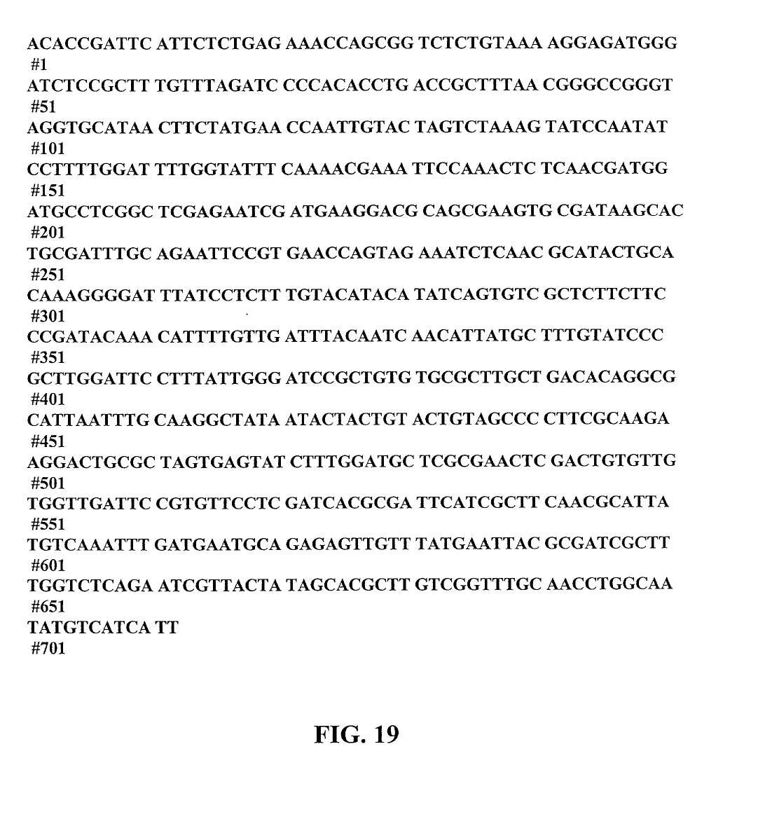

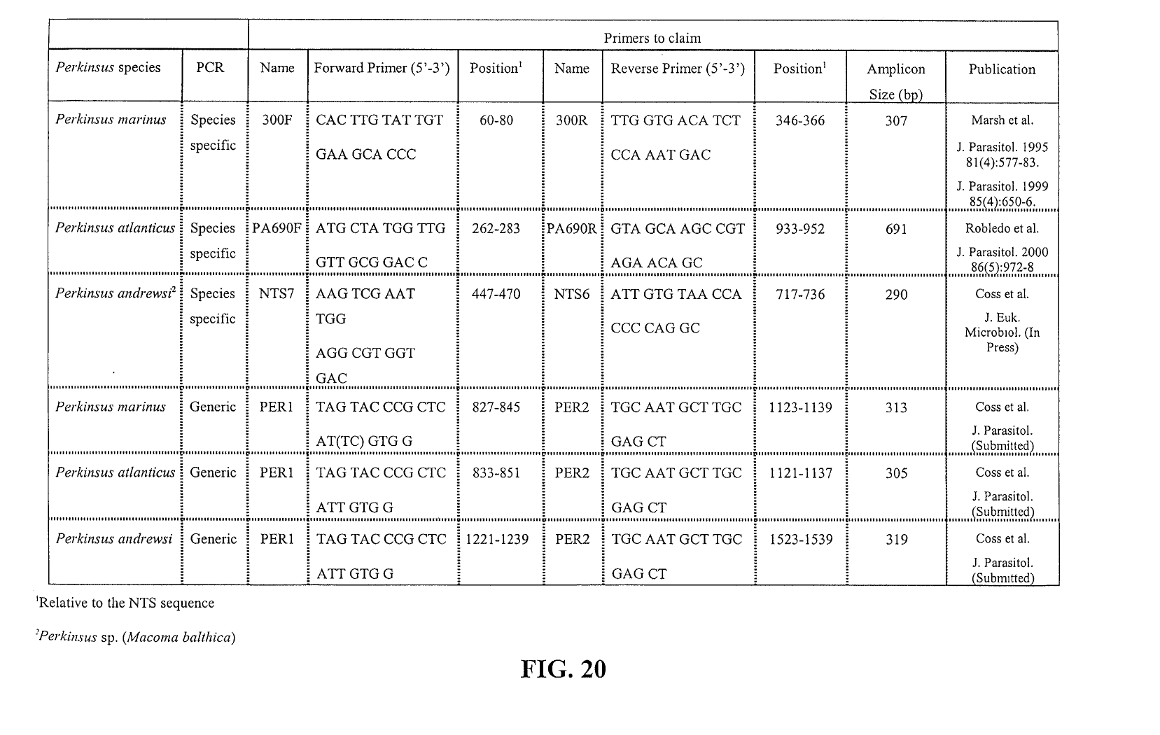

As further described in the following examples, the sequence of NTS region from P. atlanticus isolated from Galicia, Spain, has been cloned and sequences and is shown in FIG. 17. The most suitable pair of P. atlanticus-specific primers consisted of forward sequence (PA690F, 5′ ATG CTA TGG TTG GTT GCG GAC C 3′) and a reverse sequence (PA690R, 5′ GTA GCA AGC CGT AGA ACA GC 3′) that would result in amplicon of 690 bp is shown in FIG. 20. P. atlanticus DNA was not amplified by using the PCR-based assay specific for P. marinus. Some details of the method may be found in the publication, “Characterization of the ribosomal RNA locus of Perkinsus atlanticus and development of a polymerase chain reaction based diagnostic assy” 2000, Journal of Parasitology 86 (5):972-978.

Additionally, regions of the rRNA locus (NTS, 18S ITS1, 5.8S and ITS2) was isolated from the baltic clam Macoma balthica (P. andrewsi) and cloned, sequenced and compared by alignment with those available for other Perkinsus species and isolates. Primers based on P. andrewsi NTS sequence were developed and used in PCR-based diagnostic assay that was validated for species-specificity and sensitivity. PCR-based assays specific for either P. andrewsi or P. marinus were used to test for their presence in bivalve species sympatrzic to M. balthica. Although isolated from M. balthica, P. andrewsi was also detected in the oyster Crassostrea virginica and clams Macoma mitchelli and Mercenaria mercenaria, and could co exist with P. marinus in all four bivalve speices tested.

C. Using the Assay

To conduct the assay a DNA sample is extracted from any tissue or body fluid of the shellfish. DNA is extracted using conventional techniques such as described in Sambrook et al. (1989). Target DNA is amplified by adding a pair of outwardly-directed primers (made as described above), wherein the primers can hybridize to the NTS sequences, separating the extension products generated in the amplification step by size, and the specific species and strain of Perkinsus determined by sequence or enzymatic digestion of the extension products.

In addition to the PCR-diagnostic assay, the NTS region can be used to develop a quantitative PCR assay retaining specificity that will permit the accurate assessment of the numbers of P. marinus in tissue and hemolymph of infected oysters. For a number of applications and studies, it is essential to determine accurately the number of parasites in different samples. Competitive PCR offers a precise method for determination of the concentration of target molecules which can than be calibrated to calculate cell number. The basis for competitive PCR is the design of a competitor template whose product can be distinguished from experimental template but at the same time is extremely similar in its composition. This competitor template is added to the PCR reaction is known quantities and co-amplified with sample DNA and the ratio of known amount of competitor product to experimental product can be used to determine the DNA concentration of the experimental template and correlate the amount of template produced with a standard cell number. Kits available on the market (PCR Mimic System, Clontech, Palo Alto, Calif.) can be used to construct competitive fragments for quantitative PCR.

Since the NTS region has resulted in the ideal choice for diagnostic intent, a series of techniques now available can be applied using this region a base, for example, in situ detection of PCR-amplified DNA. This technique combines the cell localizing ability of in situ hybridization with the extreme sensitivity of PCR. Although PCR is a faster technique than FTM, significant reduction of time can be achieved by adapting the capillary PCR. This technique uses capillary tubes instead of microfuge tubes in combination with Rapidcycler (Idaho Technology) and PCR that usually takes between 2-4 hours can be reduced to 15 minutes. Partial or complete, the sequence of the NTS can be labeled for detect, quantitate and isolated specific polynucleotides. Both radioactive and nonradioactive labeling methods using ³²P, ³⁵S, biotin and dioxigenin are suitable to label the probe.

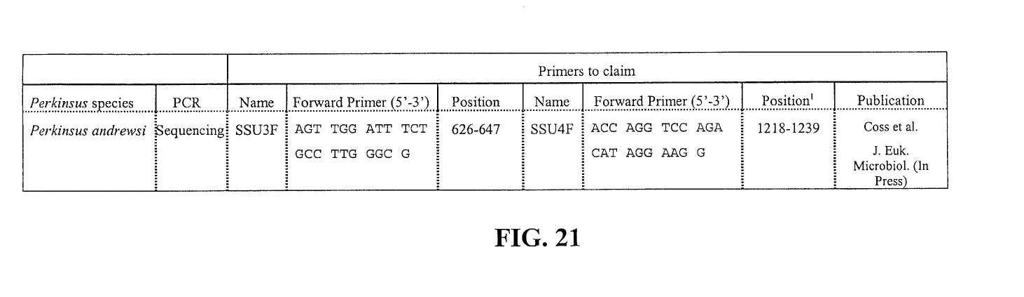

The method according to the present invention may be used to detect and distinguish among most species of organisms (pathogens or non-pathogens). In the examples herein the NTS is used to develop a PCR-based assay for several different Perkinsus species affecting oysters and clams. These NTS sequences are shown in FIGS. 2-4. It has been shown that the clams (Macoma balthica and Mercenaria mercenaria) harbor both Perkinsus marinus and Perkinsus species that are not P. marinus. This situation may also occur in oysters where parasite presence is usually assessed by either FTM assay or morphology, two techniques that do not permit specific identification of Perkinsus. The NTS regions of the Perkinsus species affecting clams have been completely sequenced and the sequence used for developing new specific primers for these Perkinsus isolates or species that will allow us to distinguish between these isolates and establish whether oysters have been exposed to multiple Perkinsus species infections (see FIGS. 20-21). Other future uses include the employment of the NTS for developing a diagnostic assay for Vibrio vulnificus, a serious human pathogen that oysters, as filter feeders, can accumulate in their tissues.

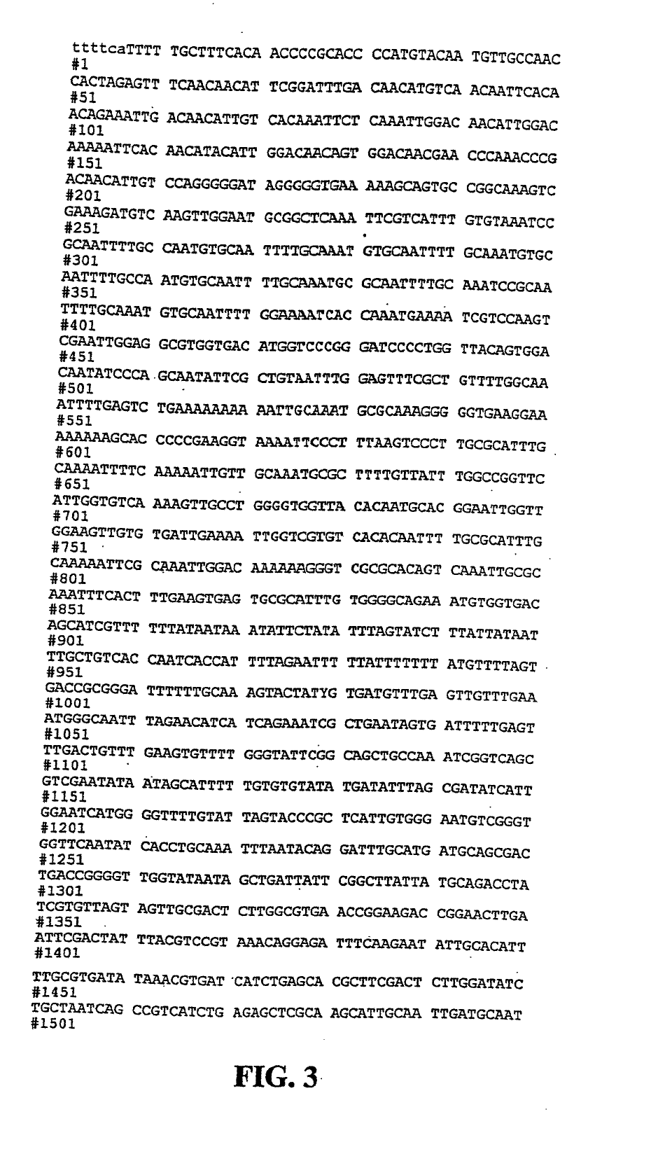

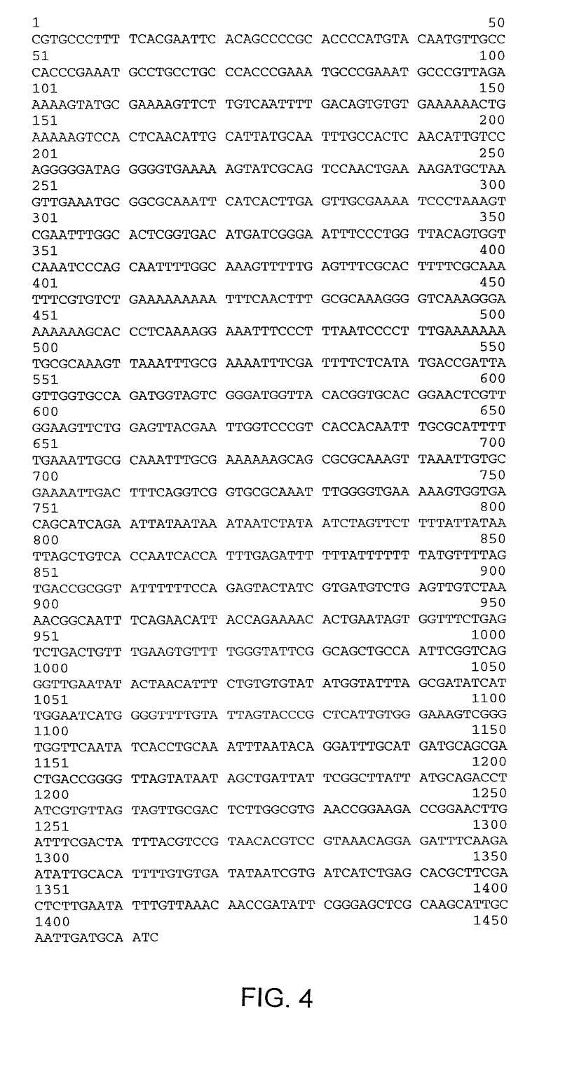

In a preferred embodiment five sets of primers are used in PCR amplification (FIGS. 20-21). The entire nontranscribed sequences of Perkinsus species number 2 from Macoma balthica, (P. andrewsi) and Perkinsus species number 3 from Mercenaria mercenaria are shown in FIGS. 3 and 4. Additional primers, of lengths greater or less that those described here, derived from this sequence could also function in the diagnostic test for Perkinsus species described herein.

Samples were also tested using a new set of primers, referred to as Perkinsus-“generic” primers, based on NTS sequence that amplifies P. marinus, P. altanticus, and P. andrewsi DNA (see FIG. 20).

A further embodiment of the present invention is a machine for identifying a strain of pathogen comprising an automated PCR amplifying means, a separation means, a sampling means for removing the extension products from the PCR means and transferring them to the separation means, a reading means for measuring patterns of extension products after separation of the separation means, a computer means for recording the results of the reading means and for outputting the pattern of and identifying the strain of the microorganism.

A number of automated PCR amplifying means are known on the market. For instance a thermal cycler can be used. There are a number of arms or robotic devices and other automatic pipette and sampling machines which can be used as a sampling means for removing the extension products from the PCR reaction at the appropriate times and transferring the sample for either chromatography, gel or capillary electrophoresis, mass spectrometry or other methods or techniques used separate the samples. In the preferred embodiment the separator means is regulated by the computer. After the separation the reader means is used to measure the pattern. The reader means will depend on the type of separation which is being used. For instance a wavelength densitometer reader or a fluorescence reader can be used depending on the label being detected. A radioisotope detector can be used for radioisotope labeled primers. In mass spectrometry the ions are detected in the spectrometer. A gel can be stained and read with a densitometer. The computer regulates the automated PCR amplification procedure, the sampling and removal from PCR, the automatic separation and reading of the samples and can be used to interpret the results and output the data.

The products, methods, instruments and procedures described herein can be used for a variety of purposes. Because of the sensitivity and specificity of the test one skilled in the art will readily recognize uses for this methodology. What follows is not an inclusive list of uses but only a sampling of specific areas where a current need exists for a quick and reliable test.

One important use of the present invention is certification of disease-free larvae, spat and juvenile oysters. Although during the last 25 years a significant progress in understanding this disease has been done (Perkins, 1996), fundamental aspects of the life cycle remain unclear or unknown. Such is the case of which is the life stage of the oyster that is sensitive to the onset of this disease and which is the infective stage of P. marinus in natural conditions. Many parasites establish latent and persistent infections that may pose diagnostic dilemmas. One of the main strategies to avoid the spread of P. marinus is to transplant only disease-free oysters. During many years oyster managers have depended on movement of oyster from seed areas to growing areas in order to avoid the overcrowding and to distribute the harvest geographically. The PCR assay developed is a specific, sensitive and rapid method for certifying P. marinus-free oyster seed and juveniles. In addition it may provide a tool for better evaluating and predicting the condition of oyster stocks and beds.

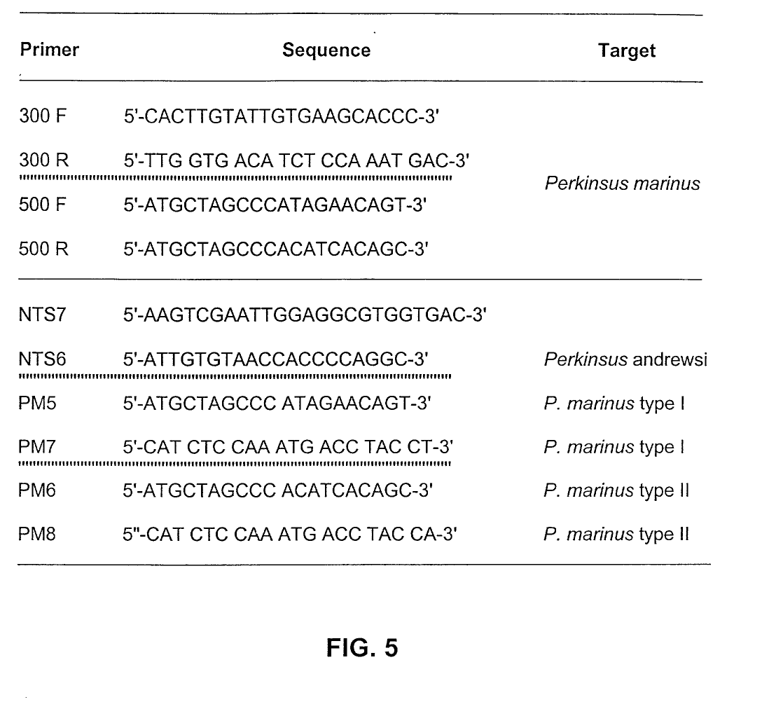

Another important use of the present invention is a kit for detecting P. marinus, P. andrewsi, and P. atlanticus and strains. The specific primers described here can be incorporated into a kit for detection of P. marinus and other Perkinsus species at various stages of oyster development. The rapid amplification of large numbers of samples may be analyzed to determine variation in population densities in environmental samples or to assay infection intensities from a large group of experimentally infected oysters. This kit preferably comprises a container having a pair of outwardly-directed PCR primers to the NTS region of the microorganism(s) being tested for. This kit can have any of the PCR primers listed in FIG. 5 or a combination thereof. One skilled in the art will readily recognize that the number and type of primers which are in the kit will depend on the use of the kit as well as the sequences to be detected. The kit would also include the buffers, DNA polymerase, and dideoxynucleotides, KCl₂ and MgCl₂ and all other reagents necessary to conduct PCR amplification. Also included would be instructions as to how to dilute the sample in preparation for “Dilution Endpoint” PCR analysis. Directions for performing the analysis by either dot blot or Southern blot hybridizations could also be included. The kit will include competitor template whose product can be distinguished from the experimental template but at the same time is extremely similar and competitor in preparation for competitive PCR.

The present invention can be used, for example, with oysters and associated invertebrate fauna from the Chesapeake Bay. The application of PCR methodology for the detection of the parasite in other shellfish species should provide information about the possibility that oysters from the Chesapeake Bay are infected by the same P. marinus type that may be present in putative reservoirs or alternative hosts. One example is Macoma balthica a bivalve that is abundant and easily obtained in the Chesapeake Bay. In addition, other organisms living on or near oyster reefs with a known with a known history of Perkinsus infection can be tested.

The invention may be used to evaluate the presence of P. marinus, P. atlanticus, and P. andrewsi in the water column or sediments. Waterborne infection particles are expected to increase during the summer as temperature and salinity increase and oysters die from the disease dumping infective particles into the water column. With the PCR assay one can detect life stages which are undetectable and more accurately than with serological methods alone.

There are several approaches for applying nucleic acid probes to the detection of specific DNA or RNA sequences, but in developing suitable applications for P. marinus, we have selected biotechnological strategies to provide: a) a rapid assay that could be implemented in most labs with a minimum of specialized equipment; b) a species-specific assay that can be applied to any bivalve tissue for diagnostic purpose; c) a strain-specific assay that could provide genetic lineage information about a particular Perkinsus sample; d) a sensitive assay for the detection of P. marinus in tissues of oyster juveniles spat, e) the possibility to extend this diagnostic strategy for any Perkinsus species. The invention herein meets these objectives.

Any quantitative diagnostic assay requires a rigorously established detection limit. The sensitivity of the PCR assay was assessed through spike and recovery experiments using P. marinus cells in the presence of parasite tissue. The PCR-based diagnostic assay is able to detect as few as one cell in presence of 30-40 mg of oyster tissue.

From the agarose gels of Example 3b, it is apparent that there are distinct differences in the amplification intensity of the P. marinus DNA target. The most likely source of these differences is the amount of P. marinus DNA in each of the oyster sample DNA extracts. Most quantitative PCR strategies essentially involve some form of a competitive assay in which the amplification of a known template is used to calculate an efficiency that is subsequently used to convert the amplification of an unknown back to its starting template concentration (see Innis et al., 1990). These techniques all require a genetically engineered standard target and a thorough quantification of reaction kinetics.

In contrast, a semi-quantitative assay is used herein that can be performed on any sample without any prior preparation or standardization. It is based on identifying the lowest dilution at which the amplification of a specific target sequence is no longer detectable. Limiting dilution assays are routinely used for many cell biology applications, but only recently have such assays been developed for the detection sensitivity of PCR (Sykes et al., 1992). The accuracy of the assay is only as fine as the dilution level employed to titrate the Endpoint, but the precision in our samples is high and there appears to be no affect by the presence of significantly higher levels of oyster DNA. We refer to this technique as ‘Dilution EndPoint’ PCR. Estimating an infection level to the nearest power of 10 may not appear to be an accurate measure, but it may provide the degree of quantification necessary to determine changes in oyster infection levels in response to experimental manipulations.

In summary, the present invention based on the NTS from sequence comprises a PCR-based diagnostic assay for the detection and quantification of P. marinus, P. andrewsi, and P. atlanticus DNA in oyster and other bivalves DNA extracts. This technique provides a rapid and reliable assessment of Perkinsus species infection levels. The PCR assay establishes a new diagnostic procedure that provides a level of sensitivity and quantification that is not afforded by the FTM assay. This invention also comprises a PCR-based diagnostic assay for the detection of Perkinsus species. in bivalves DNA extracts.

### EXAMPLES

The present invention will now be further illustrated by, but by no means limited to, the following Examples.

### Example 1a

### Design and Preparation of Primers (Perkinsus marinus)

Total DNA was extracted from axenic cultures of P. marinus using a standard SDS/proteinase-K protocol (Ausubel et al., 1992). From a BamHI endonuclease digestion, a 3.2 Kbp fragment was gel purified and cloned into the polylinker of pBluescript (Stratagene, La Jolla, Calif.). Both strands of this clone were sequenced using dideoxy terminators on an ABI automated DNA sequencer according to the manufacturer's instructions. Sequence analysis using both GCG-FASTA searches through GenBank and PAUP alignments revealed that the 3.2-kb clone encoded the 5S and SSU rRNA genes separated by a 1.1-kb non-coding domain. The development of a PCR-based assay for this DNA fragment focused on the sequence information of the non-coding domain between the two rRNA genes. Oligonucleotide primers were designed for this region using the PRIMER program (V0.5, Whitehead Institute, Cambridge, Mass.) with stringent criteria, including a requisite that their melting temperatures be above 58° C. The best pair of primers was the forward sequence 5′-CAC TTG TAT TGT GAA GCA CCC-3′ and the reverse sequence 5′-TTG GTG ACA TCT CCA AAT GAC-3′ which would amplify a 307 bp target region. These primers were synthesized on a Beckman Oligo 1000 DNA synthesizer, quantified by optical density at 260 nm, and diluted to 100 μM working stock solutions with sterile water.

Oysters were obtained from three sources. One dozen oysters were purchased from Mook Sea Farms, Damariscotta, Me., to serve as negative (uninfected) controls. Fourteen oysters were obtained from two sites in Louisiana and shipped to us to serve as our primary field samples. We obtained nine DNA samples that had been prepared from oyster gill tissues from individuals collected at nine sites along the Gulf of Mexico oyster from a previous study (stage 5 of the Mackin [1962] scale for the thioglycollate assay) was extracted for use in this study as a positive infection control.

### Example 1b

### Design and Preparation of Primers (Perkinsus spp.) for P. andrewsi, P. atlanticus and Perkinsus-“generic” Primers

Total DNA was extracted from axenic cultures of Perkinsus spp. from Macoma balthica and Perkinsus spp. from Mercenaria mercenaria by adapting a spin-column methodology designs for the isolation of DNA from human tissues (QIAGEN, Valencia, Calif.). Sample optical density at 260 and 280 nm was used to quantify the DNA concentration and assess the DNA quality. PCR primers flanking NTS sequences of P. marinus in the 5.0S and SSU rRNA gene (unpublished data) were used for amplification of the NTS region of the Perkinsus isolates. PCR reactions were performed following Goggin (1994) in a total volume of 25 μl using DNA Pelticer Thermal Cycler (MJ Research) and resolved on 1.5% agarose gel in the presence of ethidium bromide (EtBr, 10 ng/ml final concentration in the gel). PCR amplification products from M. balthica and M. mercenaria were cloned into pGEM-T Vector (Promega). Recombinant plasmids were used to transform JM 109 Competent cells and were selected on Xgal, IPTG, ampicillin, tetracycline LB plates, following the manufacture's protocol. Individual colonies were grown overnight in LB or Terrific borth and minipreps extracted using the Wizard, Plus Minipreps DNA purification System (Promega). For each isolate five clones with inserts were sequenced via the dideoxy chain termination method using the DNA Sequencing Kit (Perkin Elmer, Foster City, Calif.) in an Applied Biosystems 373 DNA as described in the following example, the DNA from in vitro propagated P. andrewsi n.sp. was extracted using the QIAamp tissues kit (QIAGEN, Valencia, Calif.) following the manufacturer's instructions. The NTS, SSU, and ITS fragments were amplified following Robledo et al. (1999), Medlin et al. (1988), and Goggin (1994) respectively. At least three clones of each type from two amplification reactions for each region (NTS, SSU, and ITSI-5.8S-ITS2) were cloned, sequenced as reported elsewhere (Robledo et al. 1999), and deposited in GenBank™ (AF102171).

SSU rRNA sequences for comparison were obtained from GenBank™ including P. marinus (X75762), P. atlticus (AF140292) Perkinsus spp. from A. trapezia (L07375) and Perkinsus spp. (G117) from Marenaria (AF042707). rRNA ITS sequences for comparison were obtained from GenBank™ for P. marinus, P. olseni, P. atlanticus, P. qugwadi, and Perkinsus sp.(G117) from Mercenaria (U0770, U07701, U07697, AF151528, and AF091541). Sequences were aligned usint the “pileup” program of the Wisconsin GCG package (GCG, Madison, Wis.), and realignments were made by eye. Oligonucleotide primers for P. andrewsi were designed for this region. The best pair of primers was the forward sequence 5′-AAG TCG AAT TGG AGG CGT GGT GAC-3′ and the reverse sequence 5′-ATT GTG TAA CCA CCC CAG GC-3′.

As further described in the following example the DNA from zoosporangia and zoopsores from P. atlanticus, and clam tissues from R. decussatus, R. philippinarum, and V. pullastra were extracted by using a spin-column methodology designed for the isolation of DNA from mammalian tissues (QIAGEN, Valencia, Calif.) as reported elsewhere (Robledo et al., 1998). DNA was extracted from cultured cells of P. marinus and Perkinsus sp. (M. balthica) using the same methodology. DNA concentration and quality was estimated by optical density at 260 and 260/280 nm, respectively.

(a) DNA amplification and cloning type

The SSU fragment was amplified using the universal primers and PCR conditions reported elsewhere (Medlin et al., 1988). The fragment including the ITSI, 5.8S, and ITS2 regions was amplified according to Goggin (1994). PCR primers designed based on the P. marinus 5.0S and SSU rRNA gene sequences flanking the NTS (Robledo et al., 1999) were used for amplifications of the NTS region of P. atlanticus. PCR amplification products were cloned into pGEM®-T Vector (Promega, Madison, Wis.) ad sequenced according to the maufacturarer's protocol (Applied Biosystems Inc., Warrington, Great Britian).

The rRNA ITS sequences for P. marinus, P. olseni, and P. atlanticus, and Perkinsus species (accession numbers U07697, U07698, U07699, U07700, and U07701), and rRNA SSU region sequences for P. marinus (accession number X75762) and Perkinsus sp. from A. trapezia (accession number L07375) were obtained from Genbank (Goggin and Barker, 1993; Fong et al., 1993; Goggin, 1994). SSU and ITS sequences for P. atlanticus from Spain were deposited in Genban (accession number AF140295). Sequences were aligned using the pileup program from the Wisconsin GCG package (GCG, Madison, Wis.), and realignments were made by eye.

(b) DNA sequence and primer design

Three clones of each type from 2 amplification reactions were sequenced via the dideoxy chain-termination method using the DNA Sequencing Kit (Applied Biosystems Inc., Warrington, Great Britain) in an Applied Biosystems 373 DNA sequencer. Sequence analysis confirmed the amplification of regions of the rRNA genes of P. atlanticus. Optimal oligonucleotide primers for PCR amplification were designed for the NTS region using the GeneJockey II program (Biosoft, Cambridge, U.K.).

Based on the NTS sequence obtained and compared to the P. marinus NTS and Perkinsus sp. (M. balthica) (62.2% identity), the most suitable pair of P. atlanticus-specific primers consisted of a forward sequence (PA690F, 5′ ATG CTA TGG TTG GTT GCG GAC C 3′) and a reverse sequence (PA690R, 5′ GTA GCA AGC CGT AGA ACA GC 3′) that would result in an amplicon of 690 bp (FIG. 20).

(c) Specificity of the PCR-based assay for P. atlanticus

To characterize the specificity of the primers designed for P. atlanticus from R. decussatus, PCR amplification of DNA from P. marinus isloated from C. virginica, DNA from Perkinsus sp. isolated from M. balthica, and DNA from P. atlanticus zoosporangia was carried out. Only P. atlanticus yielded the expected 690-bp amplicon. Perkinsus atlanticus DNA was not amplified by using the PCR-based assay specific for P. marinus (data not shown).

(d) Sensitivity of the PCR-based assay for P. atlanticus

The lowest limit of detection of the PCR-based assay for P. atlanticus isloated DNA, as determined by visualization of amplicons resolved by electrophoresis on a 1.2% agarose gel stained with ethidium bromide was 0.01 amol of cloned P. atlanticus NTS DNA. As assessed by spike/recovery experiments, the presence of 1 μg of host (R. decussatus) DNA in the reaction mixture did not affect the sensitivity of the PCR assay. The lowest limit of detection of the PCR-based assay determined in the spike/recovery experiments remained at 0.01 amol of cloned P. atlanticus NTS DNA.

(e) Presence of P. atlanticus in bivalve species

The presence of P. atlanticus was assessed in bivalves (R. phillippinarum and V. pullastra) sympatric with R. decussatus by applying the developed PCR-based diagnostic assay specific for P. atlanticus. Of the 20 R. decussatus clams tested with the PCR assay, 4 were positive for P. atlanticus. None of the R. philippinarum or V. pullastra clams tested positive for P. atlanticus. All clams were negative for P. marinus, as assessed by the P. marinus-specific PCR-based assay. Additionally, a new set of primers referred to as Perkinsus-“generic” primers, (Forward primer PER 1 5′ TAG TAC CCG CTC ATT GTG G 3′ and the reverse primer PER 2-5′ TGC AAT GCT TGC GAG CT 3′-) based on NTS sequence that amplities P. marinus, P. atlanticus, and P. andrewsi DNA was designed. PCR conditions for the Perkinsus-generic PCR assay were 94° C. for 4 min and 35 cycles at 91° C. for 1 min, 51° C. for 30 sec, and 72° C. for 1 min 30 sec with final extension at 72° C. for 7 min. PCR products were resolved on 2.5-3.0% agarose gel.

Performance of the “generic” PCR-based assay for Perkinsus species

Experiments aimed at testing the performance of the Perkinsus-“generic” primers designed for the amplification of both P. marinus and Perkinsus sp. (M.b.) Resulted in 313 bp and 319 bp amplicons respectively, under the PCR conditions described above. These Perkinsus “generic” primers also amplified an approximately 300 bp amplicon for P. atlanticus DNA. A restriction site (Spe I, ACTAGT) was identified in the P. marinus NTS sequence but not in the Perkinsus sp. (M.b.) NTS sequence. PCR products obtained with primers PER 1 and PER 2 were incubated for 3 hr at 37° C. in the presence of Spe I following manufacturer's procedures (GIBCOBRL, Gaithersburg, Md.). The digested PCR fragments were resolved on an agarose gel. Consequently, species-specific restriction digest fragments can be generated to identify and distinguish P. marinus from Perkinsus sp. (M.b.) Using the “generic” PCR products.

Sensitivity of the PCR-based assay for Perkinsus sp. (M.b.) and “generic” PCR-based assay for Perkinsus species: Spike/recovery studies

The lowest limit of detection of the PCR-based assay for Perkinsus sp. (M.b.) DNA, as determined EtBr-stained amplicons resolved by electrophoresis, was 100 fg of DNA. As assessed by spike/recovery experiments, the presence of host DNA in the reaction mixture did not modify (M. balthica DNA) or only slightly modified (C. Virginica DNA) the sensitivity of the PCR assay. The lowest limit of detection of the PCR-based assay bu the spike/recovery experiments was 100 fg of Perkinsus sp. (M.b.) DNA in the presence of 1 p g of M. balthica DNA, and 1 p g of Perkinsus sp. (M.b.) DNA in the presence of 1 p g of C. virginica DNA. The lowest limit of detection of the Perkinsus-“generic” PCR-based assay was 10 fg of Perkinsus sp. (M.b.) DNA.

Based on the partial characterization of the rRNA gene cluster, a Perkinsus species is isolated from the Baltic clam M. balthica was recently described as a species distinct from P. marinus, P. atlanticus and P. olseni (Coss et al., submitted). This Perkinsus species is not only sympatric with P. marnius in the Cheaspeake Bay region but both species can coexist in the oyster C. virginica and the clams M. balthica, M. mitchelli and Mercenaria mercenaria (Coss et al., submitted).

### Example 2a

### Extraction and Purification of DNA (Oysters Tissue Samples)

Tissue samples are processed by adapting a spin-column methodology designed for the isolation of DNA from human blood samples (QIAGEN, Valencia, Calif.). The tissues are lysed in presence of sodium dodecyl sulfate (SDS), proteinase-K, and guanidinium HCl. The microscale extracts are passed through a column matrix than binds double strained DNA and washed several times with 60% buffered ethanol to remove any contaminating proteins and lipids. The DNA is eluted from the column with water in a volume of 50 μl. Sample optical density at 260 nm is used to quantify the DNA concentration and samples are then diluted using sterile water to a final concentration of 1 μg total DNA (Crassostrea virginica and Perkinsus marinus DNA).

### Example 2b

### Extraction and Purification DNA (Oysters Hemolymph Samples)

A 1 ml sample of hemolymph was removed from the adductor muscle of each oyster through a notch in the shell. The hemocytes were pelleted in a microcentrifuge and then processed by adapting a spin-column methodology designed for the rapid isolation of DNA from human blood samples (QIAGEN, Valencia, Calif.). The hemocytes were lysed in the presence of SDS, proteinase-K and guanidinium HCl. The micro-scale extracts were passed through a column matrix that binds double stranded DNA and washed several times with 60% buffered ethanol to remove any contaminating proteins and lipids. In order to set up a diagnostic PCR assay, each reaction has to use a known amount of starting template and there are several significant advantages to adapting these separation columns to produce clean hemocyte DNA extracts: 1) they do not require the use of organic solvents (phenol and chloroform) that are required by standard extraction techniques, which dramatically reduces the handling time needed to prepare each sample; 2) RNA is removed from the sample so that a separate RNase digest is not required in order to quantitate the DNA on a spectrophotometer.

### Example 2c

### Extraction and Purification of DNA (Bivalve Tissue Samples)

Tissue samples are processed by adapting a spin-column methodology designed for the isolation of DNA from mouse tail or from tissue (QIAGEN, Valencia, Calif.). The tissues are lysed in presence of buffers and proteinase K. The micorscale extracts are passed through a membrane than binds double strained DNA and washed several times with buffered ethanol to remove any contaminating proteins and lipids. The DNA is eluted from the column with water in a volume of 50 μl. Sample optical density at 260 nm is used to quantify the DNA concentration and samples are then diluted using sterile water to a final concentration of 1 μg total DNA (Bivalve and parasite DNA).

### Example 3a

### Amplification of P. marinus DNA by PCR

All samples were subjected to identical reaction conditions for PCR amplification in an Ericomp Twin-Block, water cooled thermal cycler. A heat stable Taq DNA polymerase was purchased from Promega (Madison, Wis.) and each assay used 1.5 Units of enzyme in a 25 μl volume with the manufacturer's reaction buffer. In addition, each assay contained 1.5 mM MgCl₂, 200 μM each dNTP, 2 uM each primer and 1 μl (1 μg) of template DNA. The temperature profile for the amplification was 2′@94° C., 3′@61° C. and 2′@72° C. This temperature profile was repeated for 35 cycles. Each PCR run started with a 5′@94° C. denaturation and was completed with a 20′@72° C. extension. Alternative protocols were tested to include: more DNA polymerase, more amplification cycles, higher and lower annealing temperatures, higher primer concentrations, and higher starting template concentrations, but these did not increase the assay's detection efficiency. The conditions listed above were determined to be the optimum reaction characteristics.

### Example 3b

### Amplification of P. marinus DNA by PCR

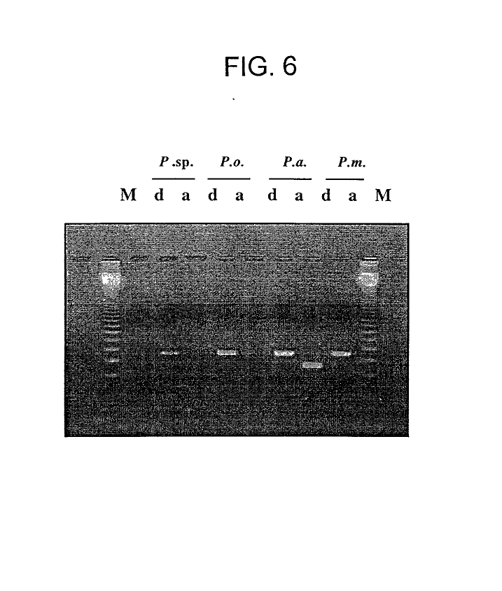

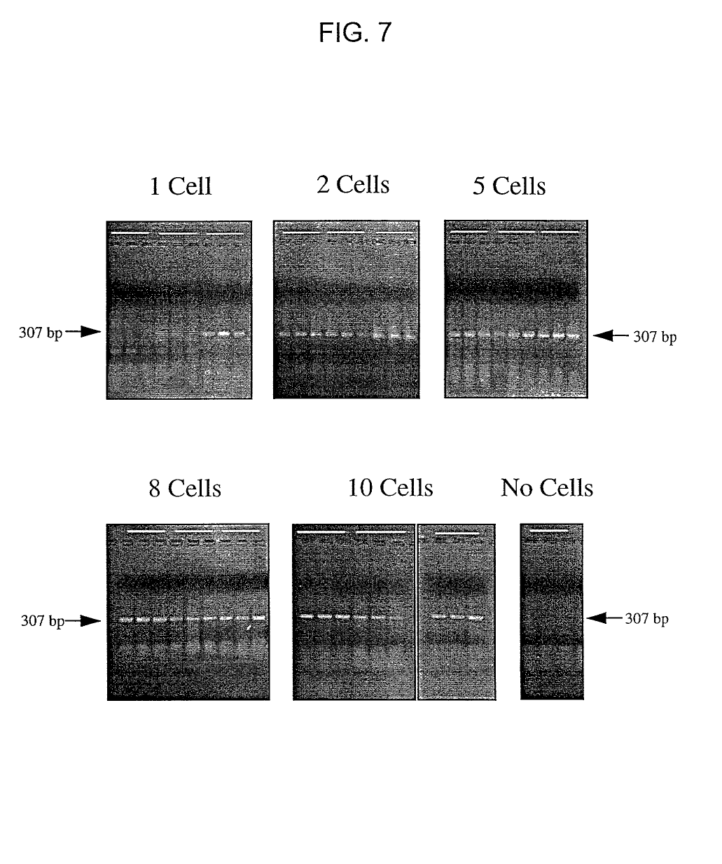

PCR primers (5′-CAC TTG TAT TGT GAA GCA CCC-3′, 300 F and 5′-TTG GTG ACA TCT CCA AAT GAC-3′, 300 R) derived from a non transcribed space (NTS) domain of rRNA sequence from P. marinus will be used for P. marinus diagnosis (Marsh et al. 1995). PCR reaction mixtures contain reaction buffer (10 mM Tris, pH 9.2; 1.5 mM MgCl₂; 75 mM KCl; 0.02% Tween-20; 10 μM TMAC; 10 μg/ml BSA; 2.5% DMSO and 5% Formamide); 1 μM of each primer; 200 μM each dATP, dCTP, dGTP and dTTP; 1.5 units of Taq DNA Polymerase (Fisher Biotech) and 1 μg DNA template in a total volume of 25 μl. Samples are heated to 91° C. for 3 min and then the reaction mixtures are cycled in a DNA Peltier Thermal Cycler (MJ Research) 35 times at 91° C. for 1 min, 58° C. for 1 min (plus 1 sec/cycle), and 72° C. for 1 min (plus 2 sec/cycle) with a final extension at 72° C. for 10 min. PCR products are resolved on a 2% agarose gel in the presence of ethidium bromide (EtBr, 10 ng/ml final concentration in the gel) and using 1× TAE buffer. A repetitive 123 bp ds DNA size standard (Promega, Madison, Wis.) is included on the gels. DNA sequencing will be by direct sequencing from PCR products and/or by cloning into a vector. This PCR is species-specific (FIG. 6) and it is able to detect as few as one cell in the presence of oyster tissue (FIG. 7). In parallel, we can apply a different set of primers to amplify a 500 bp fragment from the NTS domain.

### Example 3c

### Amplification of Perkinsus andrewsi and Perkinsus atlanticus DNA by PCR

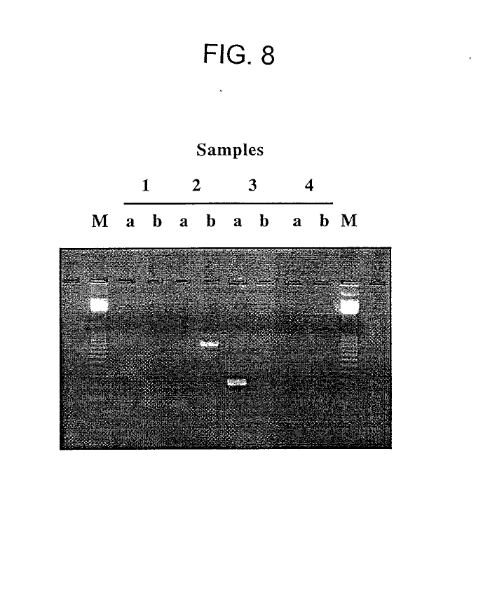

PCR primers (5′-MG TCG AAT TGG AGG CGT GGT GAC-3′, NTS7, AND 5′-ATT GTG TAA CCA CCC CAG GC-3′, NTS6) derived from a non-transcribed space (NTS) domain of rRNA sequence from Perkinsus andrewsi from Macoma balthica will be used for Perkinsus spp. diagnosis. PCR reaction mixtures contain reaction buffer (Fisher Biotech); 1 μM of each primer; 200 μM each dATP, dCTP, dGTP and dTTP; 1.5 units of Taq DNA Polymcrase (Fisher Biotech) and 1 μg DNA template in a total volume of 25 μl. Samples are heated to 94° C. for 4 min and then reaction mixtures are cycled in a DNA Peltier Thermal Cycler (MJ Research) 35 times at 91° C. for 1 min, 55° C. for 1 min (plus 1 sec/cycle), and 72° C. for 1 min (plus 2 sec/cycle) with a final extension at 72° C. for 10 min. PCR products are resolved on a 1.5-2% agarose gel in the presence of ethidium bromide (EtBr, 10 ng/ml final concentration in the gel) and using 1× TAE buffer. A repetitive 123 bp ds DNA size standard is included on the gels (FIG. 8). Alternatively new sets of primers derived from the NTS domain may be used for amplification.

### Assessment of Intraspecific Variablity in rRNA of P. andrewsi n.sp.

DNA from M. balthica collected from Chesapeake Bay (Maryland) and C. virginica from Maryland and Maine infected with P. andrewsi n.sp. were used for assessment of intraspecific variablity in selected regions of the rRNA. A 3.8 kb fragment comprised of part of the NTS, and the complete sequence of the SSU, ITS1-5.8S-ITS2 was generated using primer NTS 7 (FIG. 5), specific for P. andrewsi n.sp. (see below) and the ITSD primer of Goggin (1994). This fragment was used as a template for nested PCR with primers for the (NTS6/NTS7), expected product size 290-bp, SSU (UPRA/UPRB, Medlin, et al. 1988, expected product size 1808-bp), and ITS (ITSA/ITSD, Goggin 1994, expected product size 775-bp). These PCR products were used for direct sequence in both directions as reported elsewhere (Robledo et al. 1999). The SSU product was sequenced using additional internal primers SSU3F (Sense-5′AGT TGG ATT TCT GCC TTG GGC G-3′-) and SSU4F (Sense-5′-ACC AGG TCC AGA CAT AGG AAG G-3′-) as shown in FIG. 21.

### Development of a PCR-based Diagnostic Assay Specific for P. andrewsi n.sp.

Primers designated NTS7 (sense-5′-AAG TCG AAT TGG AGG CGT GGT GAC-3′-) and NTS6 (Antisense-5′-ATT GTG TAA CCA CCC CAG GC-3′-) were designed based on the NTS sequence using the GeneJockey II program (Biosoft, Cambridge, UK) and amplified a 290-bp fragment from P. andrewsi n.sp. DNA. PCR reaction conditions were 3 min at 94° C., 35-cycles of 94° C. for 1 min, 60° C. for 30 s, and 72° C. for 90 s, with a final extension at 72° C. for 7 min in PCR reaction mixtures as reported elsewhere (Robledo et al. 1998). Positive controls consisted of similar reaction mixtures, using 50 ng of DNA purified from cultured P. andrewsi n.sp. as template. In negative controls, template DNA was substituted by autoclaved Milli-Q-filtered water.

### Assessment of Specificity and Sensitivity of the PCR Assay for P. andrewsi n.sp.

Assay specificity was assess on 50 ng of DNA from P. marinus, P. atlanticus, and P. andrewsi n.sp. Assay sensitivity was determined directly by PCR on decreasing DNA quantities (10 ng to 10 ag) of P. andrewsi n.sp., or in a spike/recovery format using decreasing quantities of (10 ng to 10 ag) of P. andrewsi n.sp. DNA mixed with a constant amount (1 μg) of M. balthica DNA or C. virginica DNA negative for P. andrewsi n.sp. PCR reaction mixtures and conditions were as above.

### Assessment of Specificity and Sensitivity for the PCR assay for P. atlanticus

To test the specificity of the PCR-based assay for P. atlanticus, we performed the diagnostic test on DNA from P. atlanticus, P. marinus, and Perkinsus sp. from the M. balthica. Assay sensitivity was determined directly by PCR on decreasing quantities (10 amol to 0.00001 amol) of cloned P. atlanticus NTS DNA, or in a spike/recovery format in the presence of a constant amount of host clam DNA. For the latter, decreasing quantities (10 amol to 0.0001 amol) of cloned P. atlanticus NTS DNA were mixed with a constant amount (1 μg) of R. decussatus DNA negative for P. atlanticus by PCR assay. Based on the NTS sequence obtained and compared to the P. marinus NTS and Perkinsus andrewsi (62.2% identity) the most suitable pair of P. atlanticus-specific primers consisted of a forward sequence (PA 690F, 5′ ATG CTA TGG TTG GTT GCG GAC C-3′) and reverse sequence (PA 690R, 5′ GTA GCA AGC CGT AGA ACA GC3′) that would result in an amplicon of 690 bp. PCR reaction mixture contained reaction buffer (100 mM Tris, ph 9.2; 1.5 mM MgC1₂; 750 mM KCl), 0.6 μM of each primer; 200 μM each dATP, dCTP, dGTP, and dTTP; 1.5 units of Taq DNA polymerase (Fisher Biotech), and DNA template in a total volume of 25 μl. Samples were heated to 94 C. for 4 min and then the reaction mixtures were cycled in a DNA Peltier Thermal Cycler PTC 200 (MJ Research, Watertown, Mass.) 35 times at 92 C. for 1 min, 60 C. for 1 min (plus 1 sec/cycle), and 72 C. for 1 min (plus 2 sec/cycle) with a final extension at 72 C. for 7 min. PCR-based diagnosis of P. marinus was carried out as reported elsewhere (Robledo et al., 1998). For all PCR reactions, either positive controls containing cloned P. atlanticus NTS or 50 ng of P. marinus DNA, and a negative control containing no DNA template were included. PCR products were resolved as reported elsewhere (Robledo et al., 1998).

### Example 4

### Analysis of PCR Products

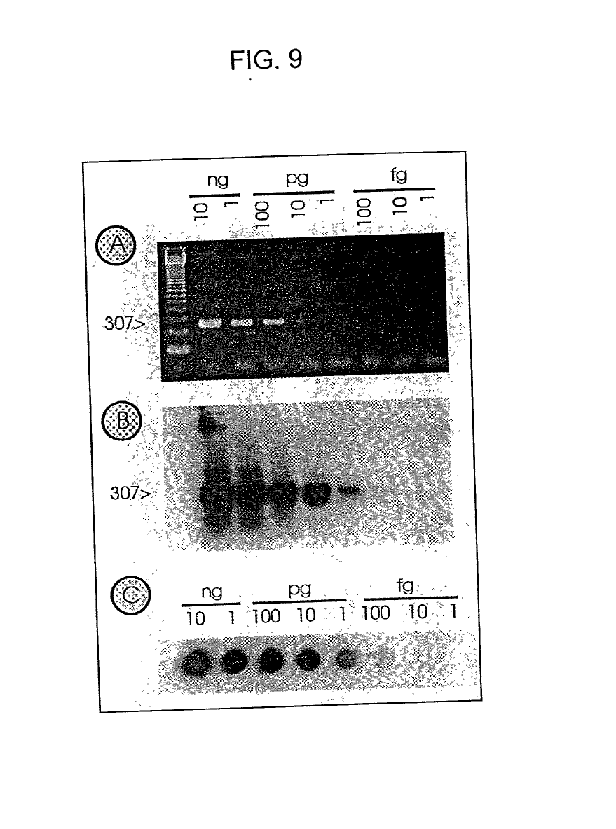

PCR products were resolved on a 1.5-2% agarose gel in the presence of ethidium bromide (EtBr; 10 ng/ml final concentration in gel) by loading 12.5 μl of the 25 μl reaction volume into each well. A repetitive 123 bp dsDNA size standard (Promega) was included on the gels (FIG. 9A). Gels were photographed and then denatured in 0.5 N NaOH with 1.5 M NzCl for 45 min, neutralized in 1 M Tris-HCl (pH 7.2) with 1.5 M NaCl for 45 min, and blotted on nylon membranes (Schleicher and Schuell, Keene, N.H.) was UV cross-linked and the membranes stored dry at room temperature. Membranes were prehybridizied for several hours in 40% formamide, 25 mN Na-PO4 (pH 7.2), 5× standard saline citrates, 0.1% SDS, 5× Denhardt's, and 50 μg/ml yeast RNA at 42° C. in a hybridization oven. A PCR amplified product with cx³²P-dCTP (3,000 Ci/mol), added to the hybridization tube with a fresh 10 ml aliquot of hybridization buffer (as above) and incubated overnight at 42° C. All PCR amplifications were first resolved on 2% agarose gels to ensure that spurius reaction products were not present (FIG. 9B). After this visual inspection, 12.5 μl aliquots of each PCR amplification were directly loaded onto nylon membranes using a dot-blot apparatus with gentle vacuum. The membranes were then denatured and neutralized as described for the agarose gels int he above section, and the DNA UV cross-linked. Hybridization conditions followed the procedure described above for the southern hybridizations (FIG. 9C).

Kodak Biomax film was used for all radiographic exposures because of the low background interference from having emulsion on only one side of the film. The oprimum length of time for exposing the film was between 12 and 24 hours with intensifying screens at 80° C. For grain densitometry, autoradiographs were digitized on a Microtek gray-scale scanner at 300 dpi and imported as TIFF files into Adobe Photoshop®. The Histogram routine in Photoshop® was used to estimate the average pixel value (white=0, black=225) for a gel band or dot-blot, which is here reported as autoradiograph grain density.

### Example 5

### Determination of P. marinus Types

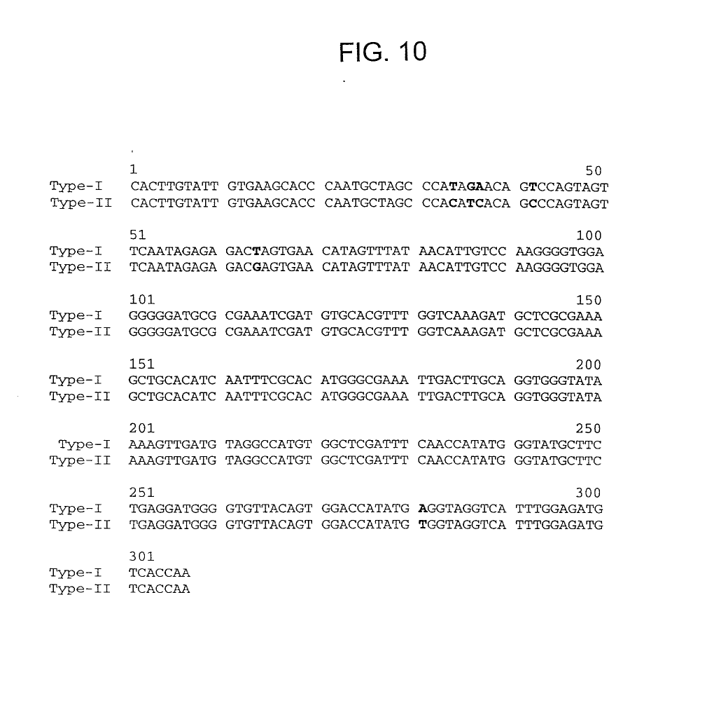

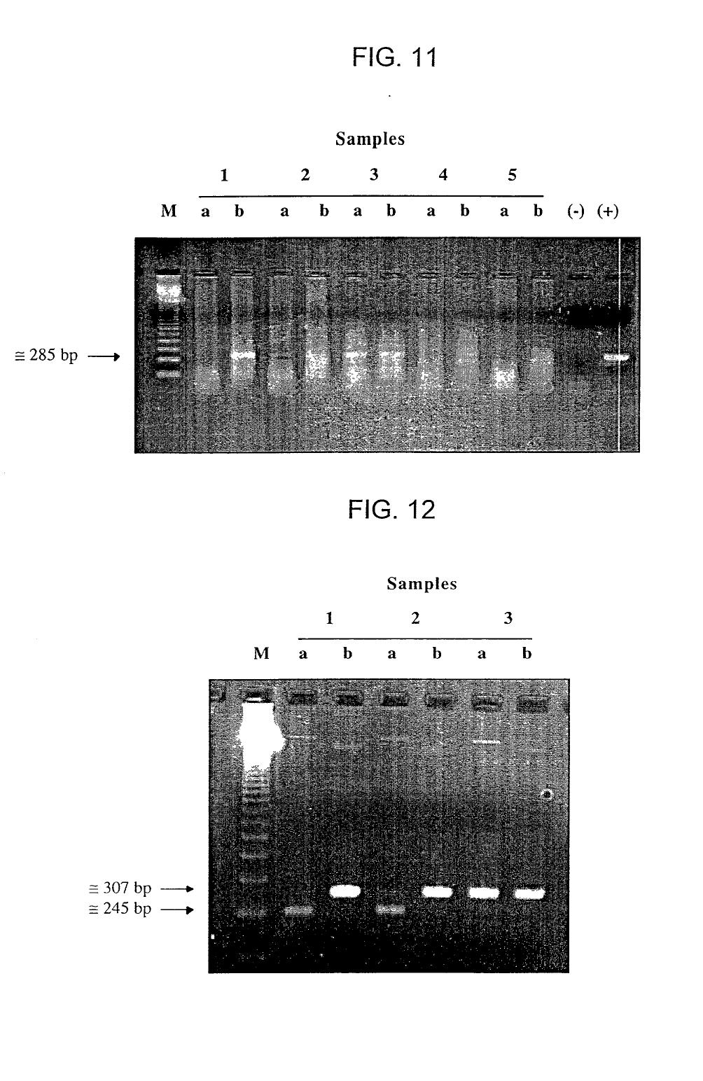

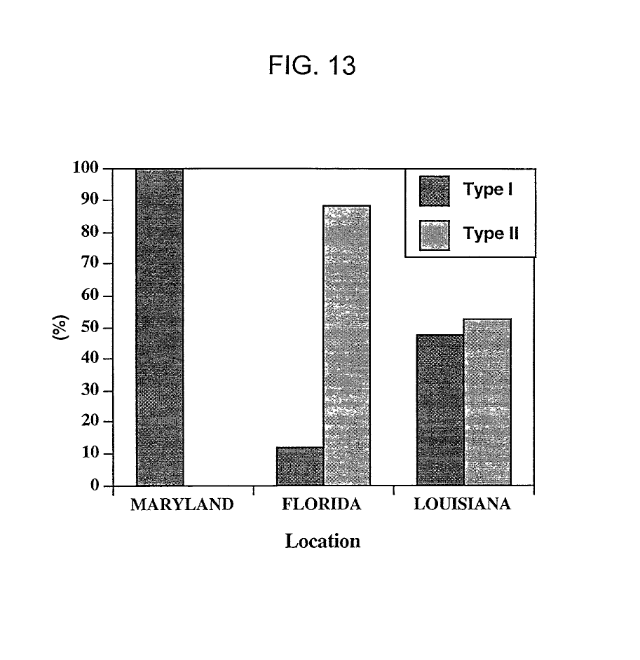

In order to identify Perkinsus type, two methods of differentiation can be applied: (a) by PCR, using newly designed sets of primers with specific amplification of individual P. marinus types and (b) restriction mapping. PCR using the PM5/PM7 primers amplified P. marinus type I and PM6/PM8 primers amplifies P. marinus type II (FIG. 10) exclusively, thus establishing specificity of the primers. The PCR reaction mixture used with the new primers was as above (Example 3B). The annealing temperature was 60° C. instead of 58° C. as used for the PCR diagnostic assay in order to increase the specificity (FIG. 11). The original diagnostic primers (not type-specifies) produced a 307 bp PCR product digestible with Spel in the case of Type I, whose sequence contains the restriction site, but not Type II, whose sequence does not hace the site. Restriction enzyme digestion was carried out using the Spel (ACTAGT) enzyme. The enzyme mix was added to a final volume of 20 μl following the manufacture recommendations (GIBCOBRL) in the presence of 200 ng of PCR products. After 3 h of incubations at 37° C., the digested products were run on a 1.5% agarose gel in the presence of ethidium bromide to resolve digested PCR fragments. One band was 245 bp and the other 62 bp (FIG. 12). Consequently, both specific PCR and restriction digestion can be used in the future for P. marinus type identification. In vitro culture methods will permit investigation of other genes that probably are more relevant for the virulence and pathogenicity of P. marinus. Restriction maps will also permit the identification of specific regions of the P. marinus genome that vary between types and specific genes present in only one type, possibly relevant to virulence and pathogenicity.

### Example 6

### Distribution of P. marinus Types in Oyster Samples

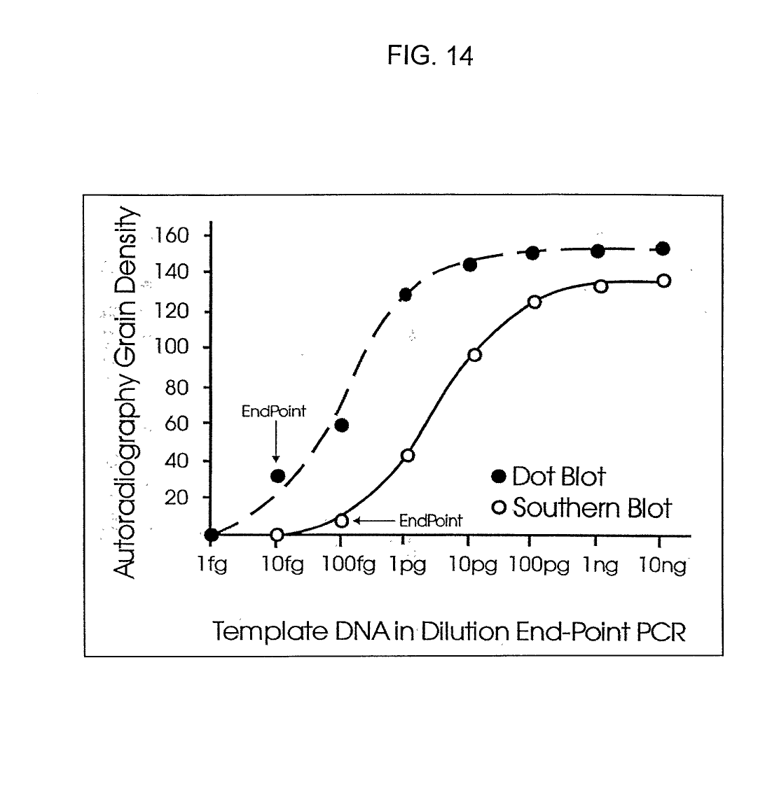

The use of the NTS domain from P. marinus rRNA gene to investigate the divergence between P. marinus affecting Crassostrea virginica has yielded the following results: 1) both types were found (types I and II) in the studied oysters (FIG. 10). 2) interestingly, the isolates from the sampled areas (Maryland, Florida and Louisiana) showed different frequencies for the P. marinus types (FIG. 13).

Analysis of P. marinus NTS sequences revealed 2 distinct sequence patterns, designated as type I and type II (FIG. 10). Both type I and type II sequences exhibited a 1-nucleotide difference (position 159) with the published sequence for this region of the P. marinus RNA locus (Marsh et al., 1995). Revaluation of the original data used for the published sequence revealed a sequencing error at this position, and therefore, the revised sequence is identical to the type I. Within the 307-bp fragment amplified, the type I and the type II sequences differed at 6 positions (base 34, 36, 37, 42, 64, and 281). Interestingly, most of the sequence dissimilarities (4 positions: 34, 36, 37, and 42) occurred in a 9-nucleotide-long region, in which the 2 sequences exhibited only 55.5% nucleotide identity. Nucleotide sequence of PCR products from in vitro-cultured P. marinus isolates from C. virginica sampled in Texas and North Carolina revealed either type I (Texas), type II (North Carolina), or a mixture of both sequence patterns (North Carolina). A P. marinus isolate from North Carolina that initially exhibited both type 1 and type II NTS sequences was subsequently resolved by limiting dilution, into monoclonal cultures that yielded either type I or type II sequences.

We suggest that this variability may reflect different P. marinus types or races as well as a new way to define the parasite distribution. We are currently identifying genetic polymorphisms in P. marinus population structure along the Gulf of Mexico and the Atlantic seaboard. We will be able to discern whether or not the genetic discontinuities that may characterize oyster populations throughout its Gulf and Atlantic coast range, are also present in P. marinus populations. The non-coding DNA domain located between the 5S and SSU rRNA genes on the 32 kb genetic element should provide us with the highest degree of interpopulational variability that is possible to detect. Establishing whether or not P. marinus has a similar or greater capacity for water-column dispersal or its presence in alternative hosts or reservoirs will be an important consideration in developing sampling strategies to look for geographic strains or races of P. marinus.

### Example 7

### Development of a ‘Dilution Endpoint’ for P. marinus Quantification

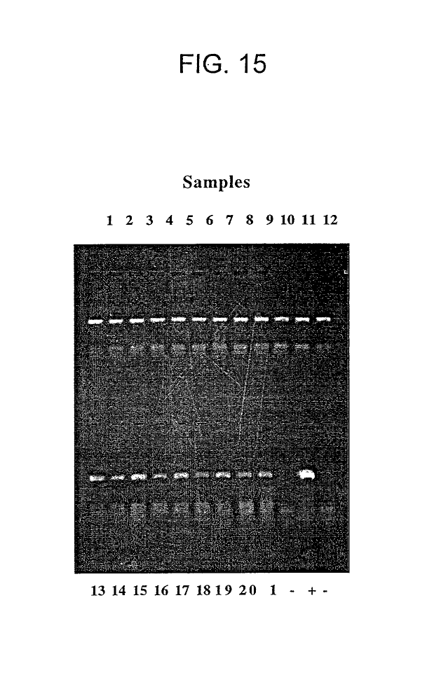

In order to estimate the amount P. marinus DNA from oyster and environmental samples a semi-quantitative methodology was developed based on the PCR that specifically target the NTS region. This method relies on determining the lowest dilution level that is necessary to distinguish any amplification of target by PCR. Because there was no detectable difference either with or without the presence of oyster DNA in the standard-diluted 10-fold with water. A 1 μl aliquot of each dilution was then used as template in PCR amplifications. Reaction products were dot-blot hybridization signal could no longer be distinguished from the background signal. By assigning a value of ‘1’ to the dilution level at which the amplification signal was extinguished, a titer for P. marinus DNA could be estimated for each preceding dilution (FIG. 14). The titer curves for the unknown samples evidence similar sigmoidal saturation kinetics as the standards, demonstrating that the amplification kinetics between the two are identical.

The ‘Dilution Endpoint’ PCR amplifications thus provide a semi-quantitative estimate ( to the nearest power of 10 in this case) of the initial concentration of P. marinus DNA in the oyster hemolymph extracts.

### Example 8

### Demonstration That Methodology Will Detect P. marinus DNA in Oyster Samples

Adult commercial oysters (Crassostrea virgincia) were collected in 1994 from Tred Avon River in Maryland (n=24) and from Bay Tambour in Louisiana (n=20). A 1.5 ml sample of hemolymph was withdrawn from the adductor muscle through a notch in the shell using a 21-gauge needle. After hemolymph extraction oysters were opened, dissected under microscopy and a 10-20 mg section of mantle tissue from the areas surrounding both labial palpes and rectum was removed. An equivalent section of rectal tissue was also taken. Samples were used for Perkinsus marinus screening using the PCR -based assay developed by Marsh et al. (1995) for P. marinus.

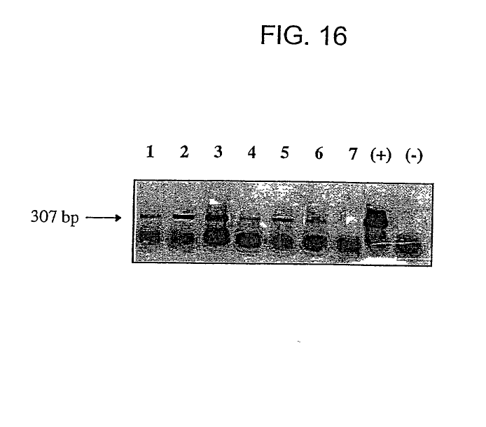

Hemolymph and tissue samples were processed as in Example 2a and 2b respectively. PCR amplification was performed following the protocol described in Example 3b. The PCR was revealed as very accurate and sensible technique for P. marinus diagnostic (FIG. 15). Oysters from Maryland, 23 templates (4 from hemocytes, 7 from rectum, 12 from mantle) that were negative by FTM, became positive with PCR for most of the tissues analyzed. There were two templates from hemocytes that were negative by FTM that with PCR became positive.

### Example 9

### Demonstration That Methodology Will Detect P. marinus DNA in Clam Samples

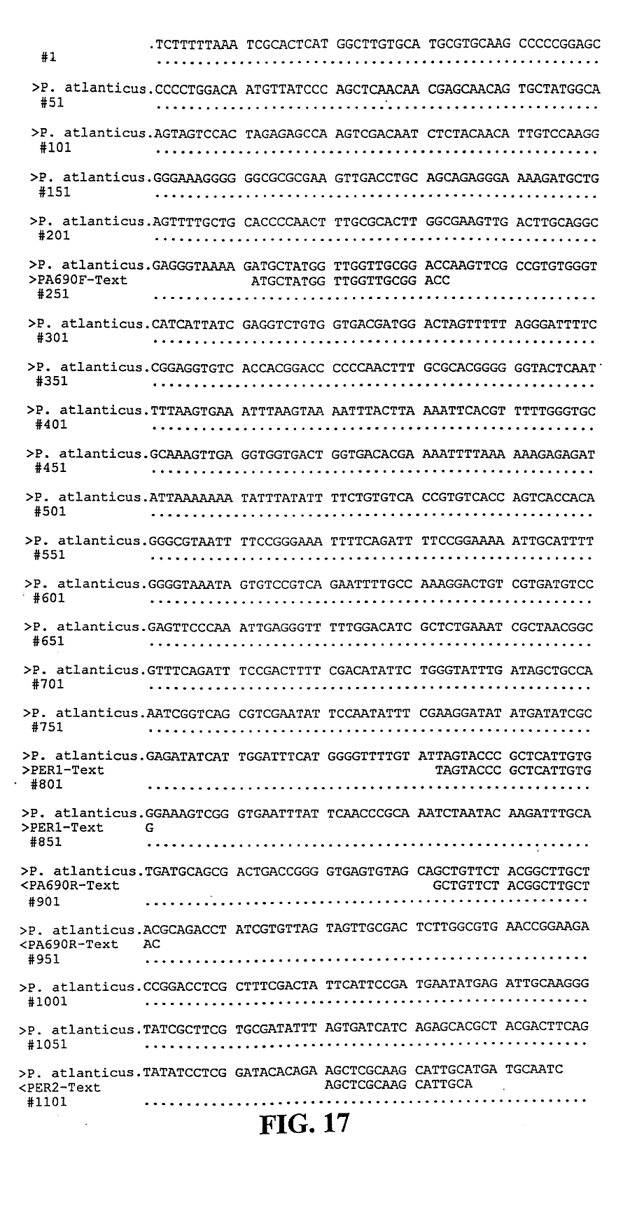

Adult clams (Macoma balthica and Mercenaria mercenaria) were obtained from the Rhode River in Chesapeake Bay and from the Indian River in Delaware Bay. PCR conditions were as in example 3b. The PCR based assay developed to detect P. marinus in oysters, was able to detect the same parasite in clam (M. balthica) (FIG. 16).

Although certain presently preferred embodiments of the invention have been described herein, it will be apparent to those skilled in the art to which the invention pertains that variations and modifications of the described embodiment may be made without departing from the spirit and scope of the invention. Accordingly, it is intended that the invention be limited only to the extent required by the appended claims and the applicable rule of law.

What is claimed is:

1. An oligonucleotide which hybridizes to a non-transcribed spacer sequence between rRNA genes of an organism of the genus Perkinsus being assayed, wherein said organism of genus Perkinsus contains a nucleotide base sequence selected from the group consisting of the sequences shown in FIGS. 2,3,4 and 17.

2. A method of making an oligonucletide for use in assaying a target organism of the genus Perkinsus comprising the steps of:
(i) extracting DNA from said target organism
(ii) isolating from said DNA a non-transcribed spacer sequence flanked by rRNA genes;
(iii) sequencing said non-transcribed spacer sequence; and
(iv) synthesizing and oligonucleotide having a nucleic acid sequence as shown in FIG. 17.

3. A kit for determining the identity of species of a microorganism of the genus Perkinsus, comprising a container having outwardly directed PCR primer pairs to a nontranscribed spacer sequence flanked by rRNA genes, said primer pairs, having a nucleic acid sequence selected from the group consisting of sequences shown in FIGS. 2,3,4 and FIG. 17.

4. The oligonucleotide of claim 1 wherein said organism is Perkinsus atlanticus

5. The oligonucleotide of claim 4 wherein said nucleotide base of said organism sequence is shown in FIG. 17.

6. The oligonucleotide of claim 1 wherein said organism is Perkinsus andrewsi

7. The oligonucleotide of claim 6, wherein siad nucleotide base sequence of said organism is shown in FIG. 3.

8. The oligonucleotide of claim 1, whrein said organism is Perkinsus mackini.

9. The oligonucleotide of claim 1 wherein said oligonucleotide is one of a pair of PCR primers, or complement thereof.

10. The oligonucleotide of claim 9, wherein said oligonucleotide is between about 10 to 35 nucleotides in length.

11. The oligonucleotide of claim 9, wherein said oligonucleotide is between about 15 to 24 nucleotides in length.

12. The oligonucleotide of claim 9 wherein said PCR primers or complement thereof are selceted from the group consisting of:
CAC TTG TAT TGT GAA GCA CCC
TTG GTG ACA TCT CCA AAT GAC
ATG CTA TGG TTG GTT GCG GAG C
GTA GCA AGC CGT AGA ACA GC
AAG TCG AAT TGG AGG CGT GGT GAC
ATT GTG TAA CCA CCC CAG GC
TAG TAC CCG CTC ATT GTG G
TGC AAT GCT TGC GAG CT
AGT TGG ATT TCT GCC TTG GGC G
ACC AGG TCC AGA CAT AGG AAG G
identifying said nontranscribed spacer sequences within said library using a probe specific for one of said rRNA genes.

18. The method of claim 2, wherein said oligonucleotide is one of an pair of PCR primers or complement thereof.

19. The kit of claim 3, wherein said microrganism is the genus Perkinsus.

20. The kit of claim 3 wherein said PCR primers pairs or complement thereof are selected from the group consisting of sequences as shown in FIGS. 20 and 21.
